(19) United States
(12) Patent Application Publication
(10) Pub. No.: US 2025/0027110 A1
(43) Pub. Date: Jan. 23, 2025

(54) COMPOSITIONS AND METHODS FOR INDUCING ESR1, PI3K, HER2, AND HER3 IMMUNE RESPONSES

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US); Parinaz ALIAHMAD, Oakland, CA (US)

(21) Appl. No.: 18/713,901

(22) PCT Filed: Nov. 28, 2022
(86) PCT No.: PCT/US22/80522

§ 371 Date: May 28, 2024

Related U.S. Application Data
(63) Continuation-in-part of application No. 17/537,199, filed on Nov. 29, 2021, now Pat. No. 11,510,975.

(51) Int. Cl. C12N 15/86; A61K 39/00; A61P 35/00; C07K 14/72; C07K 14/82; C12N 9/12

(52) U.S. Cl. CPC C12N 15/86; A61K 39/001106; A61K 39/001162; A61P 35/00; C07K 14/721; C07K 14/82; C12N 9/1205; A61K 2039/5256; A61K 2039/53; A61K 2039/55555; C12N 2770/36121; C12N 2770/36134; C12N 2770/36143; C12N 2840/203; C12Y 207/01137

(57) ABSTRACT

The present disclosure relates to the field of molecular virology, and particularly relates to nucleic acid molecules encoding a modified equine encephalitis virus viral genome or self-replicating RNA (srRNA) construct, pharmaceutical compositions containing the same, and the use of such nucleic acid molecules and compositions for production of desired products in cell cultures or in a living body. Also provided are methods for eliciting an immune response in a subject in need thereof, as well as methods for preventing and/or treating cancer.

### CROSS-REFERENCE TO RELATED APPLICATIONS

This application claims the benefit of priority to U.S. patent application Ser. No. 17/537,199, filed on Nov. 29, 2021. The disclosure of the above-referenced application is herein expressly incorporated by reference in its entirety, including any drawings.

### FIELD

The material in the accompanying Sequence Listing is hereby incorporated by reference into this application. The accompanying Sequence Listing file, named 058462-505001WO_Sequence Listing_ST26.xml, was created on Nov. 28, 2022, and is 101 KB.

### INCORPORATION OF THE SEQUENCE LISTING

The present disclosure relates to the field of molecular virology and immunology, and particularly relates to nucleic acid molecules encoding a modified equine encephalitis virus viral genome or self-replicating RNA (srRNA), pharmaceutical compositions containing the same, and the use of such nucleic acid molecules and compositions for production of desired products in cell cultures or in a living body. Also provided are methods for eliciting an immune response in a subject in need thereof, as well as methods for preventing and/or treating a health condition.

### BACKGROUND

Generation of resistance to cancer therapeutic or prevention agents is a common problem in the treatment of cancer or precancer, and, in several cases, the mechanism of resistance to the therapeutic agent is known. Resistance is often the result of changes in gene expression (over-expression or blocked expression of a protein), change in the gene by mutation, or altered sequences by altered splicing or translocation or altered activation of a protein in the cells (over-activation or blocked activation of a protein).

One method for dealing with those cancers in which such changes in gene expression, alteration, and mutation occur, has been the development of cancer vaccines. Cancer vaccines target antigens expressed by tumors, but application of these vaccines has not been as effective as once hoped due to induction of immune tolerance by chronic overexpression of the targeted protein in the absence of co-stimulatory molecules and the induction of an immunomodulatory environment. Preventative cancer vaccines may be more promising, but cancers are highly variable, with multiple genetic changes, but few truly universal changes. Thus, it is difficult to predict what antigens will be overexpressed on any specific cancer or whether an individual should be vaccinated and if so, with which antigens.

The disclosure provided here provides solutions to the problems existing with previous attempts to generate cancer vaccines and potentially offers improved methods for cancer treatment and prevention.

### SUMMARY

The present disclosure relates generally to the development of immuno-therapeutics, such as recombinant nucleic acid constructs and pharmaceutical compositions including the same for use in the prevention and management of various health conditions such as cancer. In particular, as described in greater detail below, some embodiments of the disclosure provide nucleic acid constructs containing sequences that encode a modified genome or self-replicating RNA (srRNA) of the alphavirus Eastern Equine Encephalitis virus (EEEV) where at least a portion of the nucleic acid sequence encoding the viral structural proteins of the modified EEEV genome or srRNA has been replaced by a coding sequence for a polypeptide construct comprising a) a coding sequence for estrogen receptor 1 (ESR1) or a variant thereof; b) a coding sequence for PI3K or a variant thereof; c) a coding sequence for HER2 or a variant thereof and d) a coding sequence for HER3 or a variant thereof. Also disclosed are recombinant cells that have been engineered to include one or more of the nucleic acid constructs disclosed herein, methods for producing a molecule of interest, and pharmaceutical compositions including one or more of the following: (a) a nucleic acid construct of the disclosure, (b) a recombinant cell of the disclosure, or (c) a pharmaceutical composition of the disclosure. Further provided in particular aspects of the disclosure are compositions and methods for eliciting an immune response in a subject in need thereof, and/or for the prevention and/or treatment of various health conditions, including cancers. The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

In one aspect of the disclosure, provided herein are nucleic acid constructs including a nucleic acid sequence encoding a modified Eastern Equine Encephalitis virus (EEEV) genome or srRNA, wherein at least a portion of the nucleic acid sequence encoding the viral structural proteins of the modified EEEV genome or srRNA has been replaced by a coding sequence for a polypeptide construct comprising a) a coding sequence for estrogen receptor 1 (ESR1) or a variant thereof; b) a coding sequence for PI3K or a variant thereof; c) a coding sequence for HER2 or a variant thereof, and d) a coding sequence for HER3 or a variant thereof.

In some embodiments, the modified EEEV genome or srRNA comprises no nucleic acid sequence encoding viral structural proteins.

In some embodiments, the nucleic acid sequence encoding the modified EEEV or srRNA is operably linked to a promoter sequence.

In some embodiments, the coding sequences of (a) through (d) are operably linked to one another within a single open reading frame (e.g., in a polycistronic ORF). In some embodiments, each antigen is under control of a separate promoter. In some other embodiments, all four antigens are under control of a single promoter, e.g., S26 subgenomic promoter.

In some embodiments, the coding sequences are operably linked to one another by a coding sequence for an autoproteolytic peptide or an internal ribosomal entry site (IRES). In some embodiments, the autoproteolytic peptide comprises one or more autoproteolytic cleavage sequences from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the IRES is from a Kaposi's sarcoma-associated herpesvirus (KSHV) IRES, a hepatitis virus IRES, a Pestivirus IRES, a Cripavirus IRES, a Rhopalosiphum padi virus IRES, a fibroblast growth factor IRES, a platelet-derived growth factor IRES, a vascular endothelial growth factor IRES, an insulin-like growth factor IRES, a picornavirus IRES, an encephalomyocarditis virus (EMCV) IRES, a Pim-1 IRES, a p53 IRES, an Apaf-1 IRES, a TDP2 IRES, an L-myc IRES, and a c-myc IRES.

In some embodiments, at least one of the coding sequences of (a) through (d) comprises one or more molecular alterations In some embodiments, the one or more molecular alterations are configured into a plurality of alteration cassettes arranged in tandem along the length of the coding sequence. In some embodiments, the plurality of alteration cassettes are operably linked to one another by one or more linkers.

In some embodiments, the coding sequence for an ESR1 variant in (a) comprises one or more molecular alterations that promote ligand-independent receptor activities. In some embodiments, the one or more molecular alterations comprises an activating mutation selected from the group consisting of K303R, E380Q, Y537C, Y537S, Y537N, and D538G.

In some embodiments, the coding sequence for the PI3K variant in (b) comprises one or more molecular alterations that promote ligand-independent receptor activities. In some embodiments, the one or more molecular alterations comprises an activating mutation selected from the group consisting of E542K, E545K, H1047L, and H1047R.

In some embodiments, the HER2 variant in (c) comprises a coding sequence for the extracellular domain and transmembrane domain.

In some embodiments, the HER3 variant in (d) comprises a coding sequence for a kinase-inactive HER3.

In some embodiments, the nucleic acid sequence has at least 80%, at least 85%, at least 90%, at least 95%, at least 96%, at least 97%, at least 98%, at least 99%, or 100% sequence identity to a nucleic acid sequence selected from the group consisting of SEQ ID NOS: 7-10.

In some embodiments, the coding sequence for the polypeptide construct comprises, in 5′- to 3′-direction: a) a coding sequence for a variant of PI3K comprising one or more activating molecular alterations selected from E542K, H1047L, E545K, and H1047R; b) a coding sequence for an autoproteolytic peptide P2A; c) a coding sequence for a variant of HER2 comprising its extracellular domain and transmembrane domain; d) a coding sequence for an autoproteolytic peptide P2A; e) a coding sequence for a kinase-inactive variant of HER3; f) a coding sequence for an internal ribosomal entry site (IRES); and g) a coding sequence for a variant of ESR1 comprising one or more activating molecular alterations selected from Y537C, E380Q, K303R, Y537S, D538G, and Y537N.

In one aspect, provided herein are recombinant cells including a nucleic acid construct as disclosed herein. In some embodiments, the recombinant cell is a mammalian cell or an insect cell.

In yet another aspect, provided herein are pharmaceutical compositions including a pharmaceutically acceptable excipient and a nucleic acid construct of the disclosure.

In some embodiments, the composition is formulated is formulated with a delivery vehicle into a delivery system, wherein the delivery system comprises a liposome, a viral replicon particle (VRP), a lipid-based nanoparticle (LNP), a polymer nanoparticle, a physiologic buffer, a microsphere, an immune stimulating complex (ISCOM), a conjugate of bioactive ligand, or a combination of any thereof. In some embodiments, the lipid is present in mass ratio of lipid to RNA from about 100:1 to about 4:1. In some embodiments, the lipid-based nanoparticles have an average diameter of about 25 nm to about 1000 nm. In some embodiments, the composition is formulated as a vaccine.

In another aspect, provided herein are methods for inducing an immune response or treating a health condition in a subject in need thereof. The method includes administering to the subject a composition comprising a nucleic acid construct of the disclosure. In some embodiments, the method is a method for inducing an immune response. In some embodiments, the method is a method for treating cancer. In some embodiments, the cancer is breast cancer. In some embodiments, the composition is administered to the subject individually as a single therapy (monotherapy) or as a first therapy in combination with at least one additional therapies.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is graphical representation of immunogenicity in mice used to optimize design of a mutant ESR1 antigen cassette. The X-axis lists the ordinality of ESR1 molecular alterations K303R, E380Q, Y537C, Y537S, Y537N, and D538G within the gene cassette and the use of linkers connecting these molecular alterations. The Y-axis shows the total T cell responses using peptides encoding ESR1 sequences in an ELISpot assay.

FIGS. 2A-2G are figures showing protein expression from BHK-21 cells transfected monogenic, bigenic, or tetragenic srRNAs encoding ESR1, PI3K, HER2, and HER3 from a panel of constructs having various molecular configurations and comparing them to monogenic constructs. Protein expression levels of monogenic constructs are assigned a value of “1”, and relative expression of each gene in bigenics or tetragenic configurations are compared with monogenic construct protein expression levels. FIG. 2A is an immunoblot for ESR1 protein expression. FIG. 2B is a chart showing the relative ESR1 expression based on the signal intensity of the immunoblot bands. FIG. 2C is an immunoblot for PI3K protein expression. FIG. 2D is a chart showing the relative PI3K expression based on the signal intensity of the immunoblot bands. FIG. 2E is a chart showing the relative expression of HER2 from based on the mean fluorescence intensity (MFI) quantified by fluorescence flow cytometry (FFC) after straining with an Alexa Fluor® 488 (AF488) labeled, HER2-specific antibody. FIG. 2F is a chart showing the relative expression of HER3 from based on the MFI quantified by FFC after straining with an allophycocyanin (APC) labeled, HER3-specific antibody. FIG. 2G is a spider chart summarizing the protein expression readout of ESR1, PI3K, HER2, and HER3 in the panel of constructs.

FIG. 3 is a graphical representation of T cell responses in mice administered constructs having various molecular configurations. The x-axis shows different constructs, in either monogenic, bigenic, or tetragenic form, having different ordinalities of ESR1, PI3K, HER2, and HER3. The Y-axis shows the total T cell responses to peptides encoding sequences derived from ESR1 molecular alterations, HER2, and HER3 in an ELISpot assay. PI3K responses were not measured in this experiment because it does not form responses in BALB/c mice.

FIG. 4 is a schematic of an exemplary neoantigen cassette. Peptides containing molecular alterations are separated by linkers to create a single cassette.

FIG. 5 is a graphical representation of T cell responses in mice administered constructs having different srRNA vectors and formulated in two different lipid nanoparticles, differing in the cationic lipid either LNP1 (“L1”) or LNP2 (“L2”) in the composition.

FIG. 6 is a schematic describing two types (i.e., therapeutic and prophylactic) of estrogen receptor positive breast cancer efficacy studies to model human disease.

### DETAILED DESCRIPTION OF THE DISCLOSURE

The present disclosure relates generally to nucleic acid constructs expressing variants of ESR1, PI3K, HER2, and HER3 for the purposes of both prophylactic and therapeutic treatment of human disease, such as, for example, breast cancer. These constructs address the problem with treatment modalities, such as cancer vaccines, due to the difficulty in predicting what antigens will be overexpressed on any specific cancer or whether an individual should be vaccinated and if so, with which antigens. Provided herein are, inter alia, gene expression systems with superior expression potential which are suitable for expressing a coding sequence for estrogen receptor 1 (ESR1) or a variant thereof, a coding sequence for PI3K or a variant thereof, a coding sequence for HER2 or a variant thereof, and a coding sequence for HER3 or a variant thereof, in recombinant cells. For example, some embodiments of the disclosure relate to nucleic acid constructs such as, e.g. expression constructs and vectors, containing a modified genome or srRNA of an Eastern Equine Encephalitis virus (EEEV) in which at least a portion of the nucleic acid sequence encoding the viral structural proteins of the modified EEEV genome or srRNA has been replaced by a coding sequence for a polypeptide construct comprising a coding sequence for estrogen receptor 1 (ESR1) or a variant thereof, a coding sequence for PI3K or a variant thereof, a coding sequence for HER2 or a variant thereof, and a coding sequence for HER3 or a variant thereof. Further provided are recombinant cells that are genetically engineered to include one or more of the nucleic acid molecules disclosed herein. Biomaterials and recombinant products derived from such recombinant cells are also within the scope of the application. Also provided are compositions and methods useful for eliciting an immune response or treating cancer in a subject in need thereof.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “construct” refers to a recombinant molecule, e.g., recombinant nucleic acid or polypeptide, including one or more nucleic acid sequences or amino acid sequences from heterologous sources. For example, polypeptide constructs can be chimeric polypeptide molecules in which two or more amino acid sequences of different origin are operably linked to one another in a single polypeptide construct. Similarly, nucleic acid constructs can be chimeric nucleic acid molecules in which two or more nucleic acid sequences of different origin are assembled into a single nucleic acid molecule. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Two or more nucleic acid constructs can be contained within a single nucleic acid molecule, such as a single vector, or can be containing within two or more separate nucleic acid molecules, such as two or more separate vectors.

The term “effective amount”, “therapeutically effective amount”, or “pharmaceutically effective amount” of a composition of the disclosure, e.g., nucleic acid constructs, srRNAs, recombinant cells, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “naked” as used herein when referencing nucleic acids that are substantially free of other macromolecules, such as lipids, polymers, and proteins A “naked” nucleic acid, such as a self-replicating RNA, is not formulated with other macromolecules to improve cellular uptake. Accordingly, a naked nucleic acid is not encapsulated in, absorbed on, or bound to a liposome, a microparticle, a nanoparticle, a cationic emulsion, and the like.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer) and/or one or more symptoms of the health condition. The subject can also be an individual who is diagnosed with a risk of the health condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Self-replicating RNA

As will be appreciated by the skilled artisan, the term “self-replicating RNA” refers to RNA molecule that contains all of the genetic information required for directing its own self-amplification or self-replication within a permissive cell. To direct its own replication, the srRNA generally (1) encodes polymerase, replicase, or other proteins which may interact with viral or host cell-derived proteins, nucleic acids or ribonucleoproteins to catalyze the RNA amplification process; and (2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. In some embodiments of the disclosure, an alphavirus srRNA construct generally contains the following elements: 5′ viral or defective-interfering RNA sequence(s) required in cis for replication, sequences coding for biologically active alphavirus non-structural proteins (e.g., nsP1, nsP2, nsP3, and nsP4), a subgenomic promoter (sg) for the subgenomic RNA (sgRNA), 3′ viral sequences required in cis for replication, and optionally a polyadenylate tract (poly (A)). In some instances, a subgenomic promoter (sg) that directs expression of a heterologous sequence can be included in the srRNA construct of the disclosure.

Further, the term srRNA generally refers to a molecule of positive polarity, or “message” sense, and the srRNA may be of length different from that of any known, naturally-occurring alphavirus. In some embodiments of the present disclosure, the srRNA does not contain at least a portion of the coding sequence for one or more of the alphavirus structural proteins; and/or sequences encoding structural genes can be substituted with heterologous sequences. In those instances, where the srRNA is to be packaged into a recombinant alphavirus particle, it can contain one or more sequences, so-called packaging signals, which serve to initiate interactions with alphavirus structural proteins that lead to particle formation.

The srRNA constructs of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 Kb to about 10 kb, about 6 Kb to about 9 Kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

### Eastern Equine Encephalitis virus (EEEV)

Eastern Equine Encephalitis virus (EEEV) is a mosquito-borne virus belonging to the genus Alphavirus which include a group of genetically, structurally, and serologically related viruses of the Togaviridae family. Currently, the alphavirus genus includes among others the Sindbis virus (SINV), the Semliki Forest virus (SFV), the Ross River virus (RRV), Venezuelan equine encephalitis virus (VEEV), and Eastern Equine Encephalitis virus (EEEV), which are all closely related and are able to infect various vertebrates such as mammalians, rodents, fish, avian species, and larger mammals such as humans and horses as well as invertebrates such as insects. In particular, the EEEV has been widely studied and the life cycle, mode of replication, etc., of these viruses are well characterized. More information in this regard can be found in, e.g., Corrin T. et al., Vector-Borne and Zoonotic Diseases, Vol. 21, No. 5, 2021. In addition, alphaviruses have been shown to replicate very efficiently in animal cells which makes them valuable as vectors for production of protein and nucleic acids in such cells Transmission between species and individuals occurs mainly via mosquitoes making the alphaviruses a contributor to the collection of Arboviruses- or Arthropod-Borne Viruses.

Each of these alphaviruses has a single stranded RNA genome of positive polarity enclosed in a nucleocapsid surrounded by an envelope containing viral spike proteins. Alphavirus particles are enveloped, tend to be spherical (although slightly pleomorphic), and have an isometric nucleocapsid. Alphavirus genome is single-stranded RNA of positive polarity of approximately 11-12 kb in length, comprising a 5′ cap, a 3′ poly-A tail, and two open reading frames with a first frame encoding the nonstructural proteins with enzymatic function and a second frame encoding the viral structural proteins (e.g., the capsid protein CP, E1 glycoprotein, E2 glycoprotein, E3 protein and 6K protein). For instance, EEEV possesses a single-stranded, positive-sense RNA genome of approximately 11.7 kb that is capped at the 5′ end and polyadenylated at the 3′ end. EEEV is transmitted by the bite of an infected mosquito and most spillover transmission occurs in low-lying areas with hardwood trees and swamps conducive of mosquito larvae development. As suggested by its name, EEEV can infect horses, causing fever, behavioral changes and other symptoms of encephalitis. Wild birds are the main reservoir for EEEV. However, infection is often deadly for horses.

The 5′ two-thirds of the alphavirus genome encodes a number of nonstructural proteins necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1-4) are produced as a single polyprotein and constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsP4 protein. The proteolytic maturation of P62 into E2 and E3 causes a change in the viral surface. Together the E1, E2, and sometimes E3 glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphavirus glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP proteins and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Estrogen Receptor 1 (ESR1)

Estrogens are steroidal hormones that function as the primary female sex hormone. Estrogen Receptor 1 (ESR1) encodes Estrogen Receptor alpha (ERα) and Estrogen Receptor 2 (ESR2) encodes Estrogen Receptor beta (ERβ). The biological effects of estrogen are mostly mediated by its binding and activation of ERα and ERβ, which are members of the nuclear receptor superfamily of transcription factors that are characterized by highly conserved DNA- and ligand-binding domains. Previous studies suggest that estrogen is associated with mammary tumorigenesis, ovarian and endometrial carcinogenesis. Approximately 70% of all breast cancers are classified as estrogen receptor positive (ER+); dependent upon constitutive estrogen receptor signaling. Although different classes of endocrine (anti-estrogen) therapies (including selective estrogen receptor modulators (SERMS), downregulators, and aromatase inhibitors (AIs)) are effective treatments for these cancers in adjuvant settings, approximately 50% of women will eventually relapse and die from metastatic ER+ disease. Thus, despite the advent of newer therapies (such as AIs) there remains an unrelenting rate of recurrence in ER+ breast cancer, particularly in cases where metastasis has occurred. Significantly, all patients that develop metastatic ER+ disease will progress to an endocrine therapy resistant disease. At this stage, there is no cure for ER+ breast cancer.

### Human Epidermal Growth Factor 2 (HER2)

The human epidermal growth factor receptor (HER) family, consisting of HER1 (also known as EGFR), HER2, HER3, and HER4, drives the progression of many epithelial malignancies (Roskoski R., Jr The ErbB/HER family of protein-tyrosine kinases and cancer. Pharmacol Res 2014; 79:34-74). HER2, known as Erb-B2 (erythroblastic oncogene B homolog 2), CD340 or p185, is a 185 kD oncoprotein that is encoded by the ERBB2 gene. It consists of three domains including an intracellular domain with tyrosine kinase property, a transmembrane domain and extra cellular domain. HER2 is a preferred dimerization partner for other HER proteins, such as HER3, with which it heterodimerizes. Dimerization with HER2 results in the autophosphorylation of tyrosine residues within the cytoplasmic domain of the receptors and initiates a variety of signaling pathways. HER2 has tumor promoting functions in some cancers, and amplification or over-expression of HER2 is associated with increased disease recurrence and poor prognosis. Treatment of HER2-amplified breast cancers with HER2-targeting tyrosine kinase inhibitors (TKIs) leads to an increase in HER3 expression and downstream signaling that results in therapeutic resistance.

### Human Epidermal Growth Factor 3 (HER3)

HER3, overexpressed in breast, lung, gastric, head and neck, and ovarian cancers and melanoma, is associated with poor prognosis (Takikita M et al., Membranous expression of Her3 is associated with a decreased survival in head and neck squamous cell carcinoma. J Transl Med 2011; 9:126; Chiu et al, HER-3 overexpression is prognostic of reduced breast cancer survival: A study of 4046 patients. Ann Surg 2010; 251(6):1107-16; Hayashi et al., High expression of HER3 is associated with a decreased survival in gastric cancer. Clin Cancer Res 2008; 14(23):7843-9; Giltnane et al, Quantitative multiplexed analysis of ErbB family coexpression for primary breast cancer prognosis in a large retrospective cohort. Cancer 2009; Begnami et al., Prognostic implications of altered human epidermal growth factor receptors (HERs) in gastric carcinomas: HER2 and HER3 are predictors of poor outcome. J Clin Oncol 2011; 29(22):3030-6; Reschke et al., HER3 is a determinant for poor prognosis in melanoma, Clin Cancer Res2 008; 14(16):5188-97; Lee et al., Assessment of Her-1, Her-2, and Her-3 expression and Her-2 amplification in advanced stage ovarian carcinoma. Int J Gynecol Pathol 2005) but has not been a credentialed therapeutic target, because it lacks catalytic kinase activity and is not transforming by itself. However, HER3 is thought to function as a signaling substrate for other HER proteins with which it heterodimerizes (Musgrove et al., Biological determinants of endocrine resistance in breast cancer. Nat Rev Cancer 2009; 9(9):631-43; Tovey et al., Can molecular markers predict when to implement treatment with aromatase inhibitors in invasive breast cancer? Clin Cancer Res 2005; 11(13):4835-42.

### PIK3CA

Pathologic activation of the PI3K pathway is among the most frequent signaling events associated with cellular transformation, cancer, and metastasis (Cancer Genome Atlas Network. Comprehensive molecular portraits of human breast tumours. Nature 2012; Mollon L, Aguilar A, Anderson E, et al. A systematic literature review of the prevalence of PIK3CA mutations and mutation hotspots in HR+/HER2-metastatic breast cancer. Cancer Res 2018;78:Suppl 13:1207-1207. Abstract; Goncalves MD, Hopkins BD, Cantley LC. Phosphatidylinositol 3-kinase, growth disorders, and cancer. N Engl J Med 2018;379:2052-2062). This is exemplified by the frequent activating mutations in PIK3CA and the loss of PTEN functionality in common cancers, such as those of the breast, colon, and ovaries. Approximately 40% of patients with HR-positive, HER2-negative breast cancer have activating mutations in the gene PIK3CA, inducing hyperactivation of the alpha isoform (p110α) of phosphatidylinositol 3-kinase (PI3K).

### Compositions of the Disclosure

As described in greater detail below, one aspect of the present disclosure relates to nucleic acid constructs containing sequences that encode a modified genome or srRNA of the alphavirus Eastern Equine Encephalitis virus (EEEV) where at least a portion of the nucleic acid sequence encoding the viral structural proteins of the modified EEEV genome or srRNA has been replaced by a coding sequence for a polypeptide construct comprising a) a coding sequence for estrogen receptor 1 (ESR1) or a variant thereof; b) a coding sequence for PI3K or a variant thereof; c) a coding sequence for HER2 or a variant thereof and d) a coding sequence for HER3 or a variant thereof. Also provided are recombinant cells and cell cultures that have been engineered to include a nucleic acid construct as disclosed herein.

### Nucleic Acid Constructs

As described in greater detail below, one aspect of the present disclosure relates to nucleic acid constructs including a nucleic acid sequence encoding a modified genome or srRNA of Eastern Equine Encephalitis virus (EEEV) where at least a portion of the nucleic acid sequence encoding the viral structural proteins of the modified EEEV genome or srRNA has been replaced by a coding sequence for a polypeptide construct comprising a) a coding sequence for ESR1 or a variant thereof; b) a coding sequence for PI3K or a variant thereof; c) a coding sequence for HER2 or a variant thereof and d) a coding sequence for HER3 or a variant thereof. In some embodiments, the sequence encoding the nucleic acid construct can be operably linked, e.g., placed under the control of elements required for expression (e.g., promoter sequences), which allow expression of the srRNA construct in a host cell, in a subject, or in an ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

Non-limiting exemplary embodiments of the nucleic acid constructs of the disclosure can include one or more of the following features. In some embodiments, the nucleic acid constructs include a nucleic acid sequence encoding a modified EEEV genome or srRNA, wherein the modified EEEV genome or srRNA is devoid of at least a portion of the nucleic acid sequence encoding one or more structural proteins of the unmodified EEEV genome or srRNA, e.g., the modified EEEV genome or srRNA does not include at least a portion of the coding sequence for one or more of the EEEV structural proteins CP, E1, E2, E3, and 6K. Virulent and avirulent EEEV strains are both suitable. Non-limiting examples of EEEV strains suitable for the compositions and methods of the disclosure include EEEV 792138, 783372, BeAn5122, BeAr300851, BeAr436087, C-49, FL91-4679, FL93-939, GML903836, MP-9, PE6, and V105-00210. Additional suitable EEEV strains include, but are not limited to those described in the Virus Pathogen Resource website (ViPR; which is publicly available at www.viprbrc.org/brc/vipr_genome_search.spg?method=SubmitForm&blockId=868&decorator=toga). In some embodiments, the modified EEEV genome or srRNA is derived from EEEV strain FL93-939.

Non-limiting exemplary embodiments of the nucleic acid constructs of the disclosure can include one or more of the following features. In some embodiments, the modified EEEV genome or srRNA is devoid of at least a portion of the nucleic acid sequence encoding one or more of the viral structural proteins CP, E1, E2, E3, and 6K of the unmodified EEEV genome or srRNA. In some embodiments, the modified EEEV genome or srRNA is devoid of a portion of or the entire sequence encoding CP. In some embodiments, the modified EEEV genome or srRNA is devoid of a portion of or the entire sequence encoding E1. In some embodiments, the modified EEEV genome or srRNA is devoid of a portion of or the entire sequence encoding E2. In some embodiments, the modified EEEV genome or srRNA is devoid of a portion of or the entire sequence encoding E3. In some embodiments, the modified EEEV genome or srRNA is devoid of a portion of or the entire sequence encoding 6K. In some embodiments, the modified EEEV genome or srRNA is devoid of a portion of or the entire sequence encoding a combination of CP, E1, E2, E3, and 6K. Some embodiments of the disclosure provide a modified EEEV genome or srRNA in which the coding sequence for nonstructural proteins nsP1, nsP2, nsP3, and nsP4 of the unmodified EEEV genome or srRNA is present, however at least a portion of or the entire sequence encoding one or more structural proteins (e.g., CP, E1, E2, E3, and 6K) of the EEEV genome or srRNA is absent. Some embodiments of the disclosure provide a modified EEEV genome or srRNA in which the coding sequence for nonstructural proteins nsP1, nsP2, nsP3, and nsP4 of the unmodified EEEV genome or srRNA is present, however at least a portion of or the entire sequence encoding one or more structural proteins (e.g., CP, E1, E2, E3, and 6K) of the EEEV genome or srRNA is absent.

In some embodiments, the modified viral genome or srRNA is devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul SF et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence. As described above, the present disclosure provides nucleic acid molecules and constructs which are devoid of partial or complete nucleic acid sequences encoding one or more viral structural proteins. The skilled artisan, having the benefit of the sequences as disclosed herein, can readily use all or a substantial portion of the disclosed sequences for the compositions and methods of the disclosure. Accordingly, the present application comprises the complete sequences as disclosed herein, e.g., those set forth in the accompanying Sequence Listing, as well as substantial portions of those sequences as defined above.

In some embodiments, the modified EEEV genome or srRNA is devoid of the entire sequence encoding viral structural proteins, e.g., the modified EEEV genome or srRNA includes no nucleic acid sequence encoding the structural proteins of the viral unmodified genome or srRNA.

The nucleic acid constructs of the disclosure further include a coding sequence for a polypeptide construct that replaces at least a portion of the nucleic acid sequence encoding the viral structural proteins of the modified EEEV genome or srRNA. In principle, the nucleic acid constructs disclosed herein can generally include any number of coding sequences for a polypeptide construct. In some embodiments, the nucleic acid constructs disclosed herein can include at least one, at least two, at least three, at least four, at least five, or at least six coding sequences for a polypeptide constructs. A coding sequence for a polypeptide construct can be a construct of genetic material that contains coding sequences and enough regulatory information to direct proper transcription and/or translation of the coding sequences in a cell, in vivo and/or ex vivo. The coding sequence for a polypeptide construct can be inserted into a vector for targeting to a desired host cell and/or into a subject. Accordingly, in some embodiments, the term “coding sequence for a polypeptide construct” can be used interchangeably with the term “expression construct.” In some embodiments, a coding sequence for a polypeptide construct can be a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

The nucleic acid constructs described herein include coding sequences for ESR1 or a variant thereof, a coding sequence for PI3K or a variant thereof, a coding sequence for HER2 or a variant thereof, and a coding sequence for HER3 or a variant thereof, which encode polypeptides containing epitopes that are able to elicit an immune response. The variants of ESR1, PI3K, HER2, and HER3 can encompass coding sequences for polypeptides having an amino acid sequence that is the same or essentially the same as that of the reference protein (e.g., ESR1, PI3K, HER2, or HER3) except having at least one amino acid modified, for example, deleted, inserted, or replaced, respectively. The amino acid replacement may be a conservative amino acid substitution, preferably at a non-essential amino acid residue in the protein. A “conservative amino acid substitution” is one in which the amino acid residue is replaced with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A variant of a protein may have an amino acid sequence at least about 80%, 90%, 95%, or 99%, preferably at least about 90%, more preferably at least about 95%, identical to the amino acid sequence of the protein. Preferably, a variant is a functional variant of a protein that retains the same function as the protein. The terms “variant”, when used in reference to a nucleic acid sequence, refer to a nucleic acid sequence that differs by one or more nucleotides from another, usually related nucleotide acid sequence. As such, the term “variant” can refer to a change of one or more nucleotides of a reference nucleic acid which includes the insertion of one or more new nucleotides, deletion of one or more nucleotides, and substitution of one or more existing nucleotides. A variant can also include a point mutation, multiple mutation, single nucleotide polymorphism (SNP), deletion, insertion, and translocation. Thus, variants of the coding sequences described herein include nucleic acids that encode polypeptides that can be, for example, full length, mutated, truncated, inactivated, peptide/epitopes or combinations thereof of ESR1, PI3K, HER2, and/or HER3.

The full-length amino acid sequence of ESR1 is set forth in SEQ ID NO: 1 as follows:

MTMTLHTKASGMALLHQIQGNELEPLNRPQLKIPLERPLGEVYLDS
SKPAVYNYPEGAAYEFNAAAAANAQVYGQTGLPYGPGSEAAAFGSN
GLGGFPPLNSVSPSPLMLLHPPPQLSPFLQPHGQQVPYYLENEPSG
YTVREAGPPAFYRPNSDNRRQGGRERLASTNDKGSMAMESAKETRY
CAVCNDYASGYHYGVWSCEGCKAFFKRSIQGHNDYMCPATNQCTID
KNRRKSCQACRLRKCYEVGMMKGGIRKDRRGGRMLKHKRQRDDGEG
RGEVGSAGDMRAANLWPSPLMIKRSKKNSLALSLTADQMVSALLDA
EPPILYSEYDPTRPFSEASMMGLLTNLADRELVHMINWAKRVPGFV
DLTLHDQVHLLECAWLEILMIGLVWRSMEHPGKLLFAPNLLLDRNQ
GKCVEGMVEIFDMLLATSSRFRMMNLQGEEFVCLKSIILLNSGVYT
FLSSTLKSLEEKDHIHRVLDKITDTLIHLMAKAGLTLQQQHQRLAQ
LLLILSHIRHMSNKGMEHLYSMKCKNVVPLYDLLLEMLDAHRLHAP
TSRGGASVEETDQSHLATAGSTSSHSLQKYYITGEAEGFPATV

In some embodiments, the coding sequence for ESR1 in the nucleic acid constructs described herein encodes the amino acid sequence of SEQ ID NO: 1. In some embodiments, the nucleic acid constructs of the disclosure include a nucleic acid sequence encoding an ESR1 having at least 80%, at least 85%, at least 90%, at least 95%, at least 96%, at least 97%, at least 98%, at least 99%, or 100% sequence identity to an amino acid sequence of SEQ ID NO: 1. In some embodiments, the coding sequence for ESR1 encodes smaller portions of the amino acid sequence of SEQ ID NO: 1. These smaller portions can include at least 8, 10, 12, 14, 16, 18, 20, 30 or more amino acids of SEQ ID NO: 1. Exemplary portions of ESR1 that are useful in the constructs disclosed herein include those in Table 1 below:

TABLE 1

| Amino acid sequence | SEQ ID NO |
|---|---|
| MEHLYSMKCKNVVPLCDLLLEMLDAHRLHAP | 11 |
| PGFVDLTLHDQVHLLQCAWLEILMIGLVWRS | 12 |
| AANLWPSPLMIKRSKRNSLALSLTADQMVSA | 13 |
| MEHLYSMKCKNVVPLSDLLLEMLDAHRLHAP | 14 |
| MEHLYSMKCKNVVPLYGLLLEMLDAHRLHAP | 15 |
| MEHLYSMKCKNVVPLNDLLLEMLDAHRLHAP | 16 |

In some embodiments, the nucleic acid constructs of the disclosure include a nucleic acid sequence encoding a portion of ESR1 having at least 80%, at least 85%, at least 90%, at least 95%, at least 96%, at least 97%, at least 98%, at least 99%, or 100% sequence identity to an amino acid sequence of SEQ ID NOs: 11-16.

In some embodiments, the coding sequence for a polypeptide construct in the nucleic acids described herein comprises one or more molecular alterations. Exemplary types of molecular alterations in the coding sequences described herein can be one or more of deletions, substitutions, insertion, duplications, mutations, frameshift variants, splice variants, and combinations of any thereof.

In some embodiments, the one or more molecular alterations are configured into a plurality of alteration cassettes. In some embodiments, the plurality of alteration cassettes are arranged in tandem along the length of the coding sequence. In some embodiments, the length and amino acid composition of the alteration cassettes can be optimized to achieve a desired activity or property of the coding sequence or variant thereof. In some embodiments, an alteration cassette of the plurality of alteration cassettes includes about 2 to about 50 amino acid residues, such as about 5 to about 45, about 10 to about 40, about 15 to about 30, about 20 to about 50, about 2 to about 30, about 3 to about 25, about 4 to about 20, about 5 to about 15, about 6 to about 10, about 3 to about 15, about 4 to about 10, about 5 to about 30, about 2 to about 5, about 3 to about 5, about 4 to about 8 amino acid residues. In some embodiments, an alteration cassette of the plurality of alteration cassettes includes 31 amino acid residues. In some embodiments, an alteration cassette of the plurality of alteration cassettes includes one, two, three, four, five, or more molecular alterations.

In some embodiments, the ESR1 variant described herein comprises one or more molecular alterations that promote ligand-independent receptor activities. These variants are activating mutations in the ligand binding domain of ESR1 which render the estrogen receptor insensitive to hormone therapies. In some embodiments, the one or more molecular alterations comprises an activating mutation selected from the group consisting of K303R, E380Q, Y537C, Y537S, Y537N, and D538G at positions corresponding to the amino acid sequence of SEQ ID NO: 1.

In some embodiments, the one or more molecular alterations are operably linked to one another by a linker. The linker can be a peptide linker, which joins together two adjacent alteration cassettes, as described herein. In some embodiments, the length and amino acid composition of the peptide linker sequence can be optimized to vary the orientation, flexibility, and/or proximity of the alteration cassettes relative to one another to achieve a desired activity or property of the ESR1 or ESR1 variant.

In some embodiments, a polypeptide linker includes a single-chain polypeptide sequence comprising about 1 to about 30 amino acid residues (e.g., 2, 3, 4, 5, 6, 7, 8, 9, 10, 11, 12, 13, 14, 15, 16, 17, 18, 19, 20, etc. amino acid residues). In some embodiments, a linker sequence includes about 2 to 30, about 3 to 25, about 4 to 20, about 5 to 15, about 6 to 10, about 3 to 15, about 4 to 10, about 5 to 30, about 2 to 5, about 3 to 5, about 4 to 8 amino acid residues.

In some embodiments, the length and amino acid composition of the linker polypeptide sequence can be optimized to vary the orientation, flexibility, and/or proximity of the alteration cassettes relative to one another to achieve a desired activity or property of the encoded polypeptide. In some embodiments, the orientation, flexibility, and/or proximity of the alteration cassettes relative to one another can be varied as a “tuning” tool to achieve a tuning effect that would enhance or reduce the activity of the encoded polypeptide or encoded polypeptide variant. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser) n, wherein n is an integer of one or more; and (Ser Gly Gly Gly Gly) n, wherein n is an integer of one or more. In some embodiments, the polypeptide linkers are modified such that the amino acid sequence Gly Ser Gly (GSG) (that occurs at the junction of traditional Gly/Ser linker polypeptide repeats) is not present. In some embodiments, the peptide linker includes an amino acid sequence selected from the group consisting of SEQ ID NOs: 25-29.

In some embodiments, the coding sequence for a polypeptide construct of the nucleic acid construct described herein encodes a variant of ESR1 comprising portions of the ESR1 amino acid sequence operably linked with GGGGS linkers (underlined). An exemplary amino acid sequence comprises that of SEQ ID NO: 2 as follows:

MEHLYSMKCKNVVPLCDLLLEMLDAHRLHAPGGGGSPGFVDLTLHD
QVHLLQCAWLEILMIGLVWRSGGGGSAANLWPSPLMIKRSKRNSLA
LSLTADQMVSAGGGGSMEHLYSMKCKNVVPLSDLLLEMLDAHRLHA
PGGGGSMEHLYSMKCKNVVPLYGLLLEMLDAHRLHAPGGGGSMEHL
YSMKCKNVVPLNDLLLEMLDAHRLHAPGGGGS

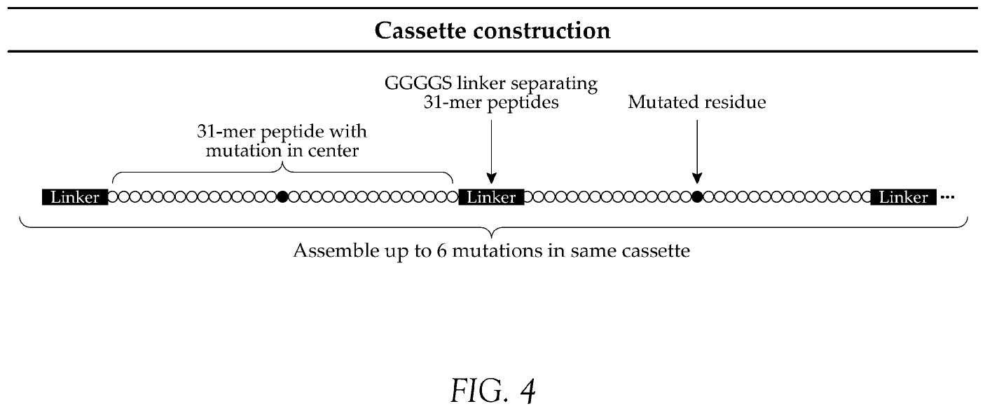

An exemplary schematic of this type of configuration is shown in FIG. 4.

In some embodiments, the nucleic acid constructs of the disclosure include a nucleic acid sequence encoding an ESR1 having at least 80%, at least 85%, at least 90%, at least 95%, at least 96%, at least 97%, at least 98%, at least 99%, or 100% sequence identity to an amino acid sequence of SEQ ID NO: 2.

As described supra, the nucleic acid constructs described herein also include coding sequences for PI3K or a variant thereof.

The full-length amino acid sequence of PI3K is set forth in SEQ ID NO: 3 as follows:

MPPRPSSGELWGIHLMPPRILVECLLPNGMIVTLECLREATLITIK
HELFKEARKYPLHQLLQDESSYIFVSVTQEAEREEFFDETRRLCDL
RLFQPFLKVIEPVGNREEKILNREIGFAIGMPVCEFDMVKDPEVQD
FRRNILNVCKEAVDLRDLNSPHSRAMYVYPPNVESSPELPKHIYNK
LDKGQIIVVIWVIVSPNNDKQKYTLKINHDCVPEQVIAEAIRKKTR
SMLLSSEQLKLCVLEYQGKYILKVCGCDEYFLEKYPLSQYKYIRSC
IMLGRMPNLMLMAKESLYSQLPMDCFTMPSYSRRISTATPYMNGET
STKSLWVINSALRIKILCATYVNVNIRDIDKIYVRTGIYHGGEPLC
DNVNTQRVPCSNPRWNEWLNYDIYIPDLPRAARLCLSICSVKGRKG
AKEEHCPLAWGNINLFDYTDTLVSGKMALNLWPVPHGLEDLLNPIG
VTGSNPNKETPCLELEFDWFSSVVKFPDMSVIEEHANWSVSREAGF
SYSHAGLSNRLARDNELRENDKEQLKAISTRDPLSEITEQEKDFLW
SHRHYCVTIPEILPKLLLSVKWNSRDEVAQMYCLVKDWPPIKPEQA
MELLDCNYPDPMVRGFAVRCLEKYLTDDKLSQYLIQLVQVLKYEQY
LDNLLVRFLLKKALTNQRIGHFFFWHLKSEMHNKTVSQRFGLLLES
YCRACGMYLKHLNRQVEAMEKLINLTDILKQEKKDETQKVQMKFLV
EQMRRPDFMDALQGFLSPLNPAHQLGNLRLEECREVISSAKRPLWL
NWENPDEVISELLFQNNEIIFKNGDDLRQDMLTLQIIREVIENIWQ
NQGLDLRMLPYGCLSIGDCVGLIEVVRNSHTIMQIQCKGGLKGALQ
FNSHTLHQWLKDKNKGEIYDAAIDLFTRSCAGYCVATFILGIGDRH
NSNIMVKDDGQLFHIDFGHFLDHKKKKFGYKRERVPFVLTQDFLIV
ISKGAQECTKTREFERFQEMCYKAYLAIRQHANLFINLFSMMLGSG
MPELQSFDDIAYIRKTLALDKTEQEALEYFMKQMNDAHHGGWTTKM
DWIFHTIKQHALN

In some embodiments, the coding sequence for PI3K in the nucleic acid constructs described herein encodes the amino acid sequence of SEQ ID NO: 3. In some embodiments, the nucleic acid constructs of the disclosure include a nucleic acid sequence encoding PI3K having at least 80%, at least 85%, at least 90%, at least 95%, at least 96%, at least 97%, at least 98%, at least 99%, or 100% sequence identity to an amino acid sequence of SEQ ID NO: 3. In some embodiments, the coding sequence for PI3K encodes smaller portions of the amino acid sequence of SEQ ID NO: 3. These smaller portions can include at least 8, 10, 12, 14, 16, 18, 20 or more amino acids of SEQ ID NO: 3. Exemplary portions of PI3K that are useful in the constructs disclosed herein include those in Table 2 below:

TABLE 2

| Amino acid sequence | SEQ ID NO |
|---|---|
| MDKEQLKAISTRDPLSKITEQEKDFLWSHRHY | 17 |
| EQEALEYFMKQMNDALHGGWTTKMDWIFHTIK | 18 |
| QLKAISTRDPLSEITKQEKDFLWSHRHYCVT | 19 |
| EQEALEYFMKQMNDARHGGWTTKMDWIFHTIK | 20 |

In some embodiments, the nucleic acid constructs of the disclosure include a nucleic acid sequence encoding a portion of PI3K having at least 80%, at least 85%, at least 90%, at least 95%, at least 96%, at least 97%, at least 98%, at least 99%, or 100% sequence identity to an amino acid sequence of SEQ ID NOs: 17-20.

In some embodiments, the coding sequence for a polypeptide construct in the nucleic acids described herein encode PI3K variants that comprise one or more molecular alterations that promote ligand-independent receptor activities. These variants are activating mutations in the ligand binding domain of PI3K. In some embodiments, the one or more molecular alterations comprises an activating mutation selected from the group consisting of E542K, E545K, H1047L, and H1047R of the amino acid sequence of SEQ ID NO: 3.

In some embodiments, the one or more molecular alterations are operably linked to one another by a linker. Linkers suitable for use in the polypeptide constructs described herein are described supra. In some embodiments, the peptide linker includes an amino acid sequence selected from the group consisting of SEQ ID NOs: 25-29.

In some embodiments, the coding sequence for a polypeptide construct of the nucleic acid construct described herein encodes a variant of PI3K comprising portions of the amino acid sequence of PI3K operably linked by GGGGS linkers (underlined). An exemplary amino acid sequence comprises that of SEQ ID NO: 4 as follows:

MDKEQLKAISTRDPLSKITEQEKDFLWSHRHYGGGGSEQEALEYFM
KQMNDALHGGWTTKMDWIFHTIKGGGGSQLKAISTRDPLSEITKQE
KDFLWSHRHYCVTGGGGSEQEALEYFMKQMNDARHGGWTTKMDWIF
HTIKGGGGS

In some embodiments, the nucleic acid constructs of the disclosure include a nucleic acid sequence encoding PI3K having at least 80%, at least 85%, at least 90%, at least 95%, at least 96%, at least 97%, at least 98%, at least 99%, or 100% sequence identity to an amino acid sequence of SEQ ID NO: 4.

As described supra, the nucleic acid constructs described herein also include coding sequences for HER2 or a variant thereof.

In some embodiments, the coding sequence for a polypeptide construct in the nucleic acids described herein encode a truncated HER2 variant that comprises the extracellular domain and transmembrane domain of HER2 as described in Crosby et al., “Vaccine-Induced Memory CD8+ T Cells Provide Clinical Benefit in HER2 Expressing Breast Cancer: A Mouse to Human Translational Study,” Clinical Cancer Research 25(9):2725-2736 (2019).

In some embodiments, the truncated HER2 variant comprises the amino acid sequence of SEQ ID NO: 5 as follows:

MELAALCRWGLLLALLPPGAASTQVCTGTDMKLRLPASPETHLDML
RHLYQGCQVVQGNLELTYLPTNASLSFLQDIQEVQGYVLIAHNQVR
QVPLQRLRIVRGTQLFEDNYALAVLDNGDPLNNTTPVTGASPGGLR
ELQLRSLTEILKGGVLIQRNPQLCYQDTILWKDIFHKNNQLALTLI
DTNRSRACHPCSPMCKGSRCWGESSEDCQSLTRTVCAGGCARCKGP
LPTDCCHEQCAAGCTGPKHSDCLACLHFNHSGICELHCPALVTYNT
DTFESMPNPEGRYTFGASCVTACPYNYLSTDVGSCTLVCPLHNQEV
TAEDGTQRCEKCSKPCARVCYGLGMEHLREVRAVTSANIQEFAGCK
KIFGSLAFLPESFDGDPASNTAPLQPEQLQVFETLEEITGYLYISA
WPDSLPDLSVFQNLQVIRGRILHNGAYSLTLQGLGISWLGLRSLRE
LGSGLALIHHNTHLCFVHTVPWDQLFRNPHQALLHTANRPEDECVG
EGLACHQLCARGHCWGPGPTQCVNCSQFLRGQECVEECRVLQGLPR
EYVNARHCLPCHPECQPQNGSVTCFGPEADQCVACAHYKDPPFCVA
RCPSGVKPDLSYMPIWKFPDEEGACQPCPINCTHSPLTSIISAVVG
ILLVVVLGVVFGILIKRRQQKIRK

In some embodiments, the nucleic acid constructs of the disclosure include a nucleic acid sequence encoding HER2 variant having at least 80%, at least 85%, at least 90%, at least 95%, at least 96%, at least 97%, at least 98%, at least 99%, or 100% sequence identity to an amino acid sequence of SEQ ID NO: 5.

As described supra, the nucleic acid constructs described herein also include coding sequences for HER3 or a variant thereof.

In some embodiments, the coding sequence for a polypeptide construct in the nucleic acids described herein encode a kinase-inactive HER3 variant as described in Osada et al., “Vaccination Targeting Human HER3 Alters the Phenotype of Infiltrating T cells and Responses to Immune Checkpoint Inhibition,” Oncoimmunology 6(6):e1315495 (2017).

In some embodiments, the kinase-inactive HER3 variant comprises the amino acid sequence of SEQ ID NO: 6 as follows:

MRANDALQVLGLLFSLARGSEVGNSQAVCPGTLNGLSVTGDAENQY
QTLYKLYERCEVVMGNLEIVLTGHNADLSFLQWIREVTGYVLVAMN
EFSTLPLPNLRVVRGTQVYDGKFAIFVMLNYNTNSSHALRQLRLTQ
LTEILSGGVYIEKNDKLCHMDTIDWRDIVRDRDAEIVVKDNGRSCP
PCHEVCKGRCWGPGSEDCQTLTKTICAPQCNGHCFGPNPNQCCHDE
CAGGCSGPQDTDCFACRHFNDSGACVPRCPQPLVYNKLTFQLEPNP
HTKYQYGGVCVASCPHNFVVDQTSCVRACPPDKMEVDKNGLKMCEP
CGGLCPKACEGTGSGSRFQTVDSSNIDGFVNCTKILGNLDFLITGL
NGDPWHKIPALDPEKLNVERTVREITGYLNIQSWPPHMHNFSVFSN
LTTIGGRSLYNRGFSLLIMKNLNVTSLGFRSLKEISAGRIYISANR
QLCYHHSLNWTKVLRGPTEERLDIKHNRPRRDCVAEGKVCDPLCSS
GGCWGPGPGQCLSCRNYSRGGVCVTHCNFLNGEPREFAHEAECFSC
HPECQPMEGTATCNGSGSDTCAQCAHFRDGPHCVSSCPHGVLGAKG
PIYKYPDVQNECRPCHENCTQGCKGPELQDCLGQTLVLIGKTHLTM
ALTVIAGLVVIFMMLGGTFLYWRGRRIQNKRAMRRYLERGESIEPL
DPSEKANKVLARIFKETELRKLKVLGSGVFGTVHKGVWIPEGESIK
IPVCIKVIEDKSGRQSFQAVTDHMLAIGSLDHAHIVRLLGLCPGSS
LQLVTQYLPLGSLLDHVRQHRGALGPQLLLNWGVQIAKGMYYLEEH
GMVHRNLAARNVLLKSPSQVQVADFGVADLLPPDDKQLLYSEAKTP
IKWMALESIHFGKYTHQSDVWSYGVTVWELMTFGAEPYAGLRLAEV
PDLLEKGERLAQPQICTIDVYMVMVKCWMIDENIRPTFKELANEFT
RMARDPPRYLVIKRESGPGIAPGPEPHGLTNKKLEEVELEPELDLD
LDLEAEEDNLATTTLGSALSLPVGTLNRPRGSQSLLSPSSGYMPMN
QGNLGESCQESAVSGSSERCPRPVSLHPMPRGCLASESSEGHVTGS
EAELQEKVSMCRSRSRSRSPRPRGDSAYHSQRHSLLTPVTPLSPPG
LEEEDVNGYVMPDTHLKGTPSSREGTLSSVGLSSVLGTEEEDEDEE
YEYMNRRRRHSPPHPPRPSSLEELGYEYMDVGSDLSASLGSTQSCP
LHPVPIMPTAGTTPDEDYEYMNRQRDGGGPGGDYAAMGACPASEQG
YEEMRAFQGPGHQAPHVHYARLKTLRSLEATDSAFDNPDYWHSRLF
PKANAQRT

In some embodiments, the nucleic acid constructs of the disclosure include a nucleic acid sequence encoding HER3 variant having at least 80%, at least 85%, at least 90%, at least 95%, at least 96%, at least 97%, at least 98%, at least 99%, or 100% sequence identity to an amino acid sequence of SEQ ID NO: 6.

In some embodiments, the coding sequence for the ESR1 or a variant thereof, PI3K or a variant thereof, HER2 or a variant thereof, and HER3 or a variant thereof includes a coding sequence for a single polypeptide (e.g., monogenic construct). In some embodiments, the coding sequence for the ESR1 or a variant thereof, PI3K or a variant thereof, HER2 or a variant thereof, and HER3 or a variant thereof includes coding sequences for a plurality of polypeptides, e.g., multigenic (e.g., bigenic or trigenic). In some embodiments, each of the coding sequences of ESR1 or a variant thereof, PI3K or a variant thereof, HER2 or a variant thereof, and HER3 or a variant thereof is operably linked to a separate promoter sequence. In some embodiments, the coding sequences of ESR1 or a variant thereof, PI3K or a variant thereof, HER2 or a variant thereof, and HER3 or a variant thereof are operably linked to one another within a single open reading frame (e.g., in a polycistronic ORF). In some embodiments, the coding sequence of the polycistronic ORF is operably linked to a promoter sequence. In some embodiments, at least one of the promoter sequences is a subgenomic (sg) promoter. In some embodiments, the sg promoter is a 26S genomic promoter.

In some embodiments, the coding sequences for ESR1 or a variant thereof, PI3K or a variant thereof, HER2 or a variant thereof, and HER3 or a variant thereof, can be linked to one another directly or indirectly (e.g., via one or more connector sequences). For example, in some embodiments, the coding sequences can be directly linked to one another, e.g., adjacently to one another. In some embodiments, at least two (e.g., 2, 3, 4, or 5) of the coding sequences are operably linked to one another by one or more connector sequences. In some embodiments, the length and amino acid composition of the connector sequences can be optimized to vary the orientation, flexibility, and/or proximity of the polypeptides relative to one another to achieve a desired activity or property of the encoded protein. In some embodiments, a connector sequence of the plurality of connector sequences includes one or more coding sequences for autoproteolytic peptide sequences. Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoproteolytic peptide” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteolytic peptides have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro, in vitro, ex vivo, and in vivo eukaryotic systems. As such, the concept of autoproteolytic peptides is available to one of skill in the art with many naturally-occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoproteolytic peptides suitable for the compositions and methods of the present disclosure include one or more autoproteolytic cleavage sequences from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequences for ESR1 or a variant thereof, PI3K or a variant thereof, HER2 or a variant thereof, and HER3 or a variant thereof are operably linked to one another by a coding sequence for one or more an internal ribosomal entry sites (IRES). An IRES or “internal ribosome entry site” is a sequence located between polycistronic genes that permits the production of the expression product originating from the second gene by internal initiation of the translation of the dicistronic mRNA. It promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In some embodiments, the IRES can be a viral IRES, a cellular IRES, or an artificial IRES. Examples of IRES generally employed by those of skill in the art include those described in U.S. Pat. No. 6,692,736. In some embodiments, the IRES is selected from a Kaposi's sarcoma-associated herpesvirus (KSHV) IRES, a hepatitis virus IRES, a Pestivirus IRES, a Cripavirus IRES, a Rhopalosiphum padi virus IRES, a fibroblast growth factor IRES, a platelet-derived growth factor IRES, a vascular endothelial growth factor IRES, an insulin-like growth factor IRES, a picornavirus IRES, an encephalomyocarditis virus (EMCV) IRES, a Pim-1 IRES, a p53 IRES, an Apaf-1 IRES, a TDP2 IRES, an L-myc IRES, and a c-myc IRES., In some embodiments, the IRES is obtainable from EMCV.

One of skill in the art will appreciate that different configurations of coding sequences for ESR1 or a variant thereof, PI3K or a variant thereof, HER2 or a variant thereof, and HER3 or a variant thereof, the sequence encoding the autoproteolytic peptide, or an IRES can be employed as long as expression of ESR1 or a variant thereof, PI3K or a variant thereof, HER2 or a variant thereof, and HER3 or a variant thereof is adequately maintained. These sequences will typically be configured so that the polypeptide encoded by the gene of interest can be released from the protease and other sequence after cleavage by the autoprotease.

The term “operably linked”, as used herein, denotes a functional linkage between two or more sequences. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

The basic techniques for operably linking two or more sequences of DNA together are familiar to one of ordinary skill in the art, and such methods have been described in many books for standard molecular biological manipulation (see, for example, Maniatis et al., “Molecular Cloning: A Laboratory Manual” 2nd ed. Cold Spring Harbor Laboratory Press, Cold Spring Harbor, N.Y.; and Gibson et al., Nature Methods 6:343-45, 2009).

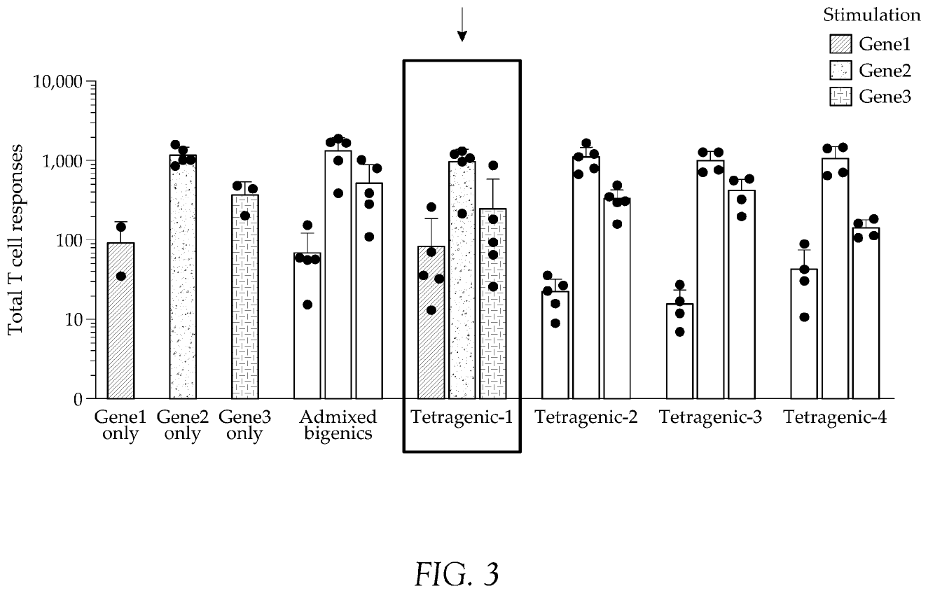

As shown in FIG. 3, T cell responses to different monogenic, bigenic, and tetragenic constructs having different ordinalities of “Gene 1”, “Gene 2”, “Gene 3”, and “Gene 4” (not shown in FIG. 3 because PI3K does not form responses in BALB/c mice) were measured. It is to be appreciate that, in some embodiments, “Gene 1”, “Gene 2”, “Gene 3”, or “Gene 4” can be ESR1 or variants thereof. Similarly, in some embodiments, “Gene 1”, “Gene 2”, “Gene 3” or “Gene 4” can be HER2 or a variant thereof. In other embodiments, “Gene 1”, “Gene 2”, “Gene 3” or “Gene 4” can be HER3 or a variant thereof. In some embodiments, “Gene 1”, “Gene 2”, “Gene 3” or “Gene 4” can be PI3K or variants thereof. Exemplary configurations of the nucleic acid constructs described herein are shown in Table 3 below.

TABLE 3

| | Final | | | |
|---|---|---|---|---|
| | ordinality | | | |
| | details | | | |
| | including | | | |
| | molecular | | | |
| | alterations | | PI3K | |
| | within | | cassette | |
| Construct ID | cassettes | ESR1 cassette ordinality | ordinality | Linker |
| pRB-136 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-146 | ESR1- | Y537C, | E542K, | GGGGS |
| | P2A- | E380Q, | H1047, | |
| | HER3- | K303R, | E545K, | |
| | P2A-PI3K- | Y537S, | H1047R | |
| | P2A-HER2 | D538G, | | |
| | | Y537N | | |
| pRB-151 | HER3- | Y537C, E380Q, | E542K, | GGGGS |
| | P2A- | K303R, Y537S, | H1047, | |
| | ESR1- | D538G, Y537N | E545K, | |
| | IRES- | | H1047R | |
| | HER2- | | | |
| | P2A-PI3K | | | |
| pRB-153 | ESR1- | Y537C, E380Q, | E542K, | GGGGS |
| | P2A- | K303R, Y537S, | H1047, | |
| | HER3- | D538G, Y537N | E545K, | |
| | IRES- | | H1047R | |
| | PI3K-P2A- | | | |
| | HER2 | | | |
| pRB-321 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-322 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-323 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-324 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-325 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | IRES- | | | |
| | ESR1 | | | |
| pRB-326 | PI3K-P2A- | Y537C, E380Q, | E542K, | GGGGS |
| | HER2- | K303R, Y537S, | H1047, | |
| | P2A- | D538G, Y537N | E545K, | |
| | HER3- | H1047R | H1047R | |
| | IRES- | | | |
| | ESR1 | | | |
| pRB-135 | ESR1- | Y537C, E380Q, | E542K, | GGGGS |
| | P2A-PI3K- | K303R, Y537S, | H1047, | |
| | P2A- | D538G, Y537N | E545K, | |
| | HER2- | | H1047R | |
| | P2A-HER3 | | | |
| pRB-137 | PI3K- | Y537C, E380Q, | E542K, | GGGGS |
| | IRES- | K303R, Y537S, | H1047, | |
| | ESR1- | D538G, Y537N | E545K, | |
| | P2A- | | H1047R | |
| | HER2- | | | |
| | P2A-HER3 | | | |
| pRB-138 | IRES- | Y537C, E380Q, | E542K, | GGGGS |
| | ESR1- | K303R, Y537S, | H1047, | |
| | P2A-PI3K- | D538G, Y537N | E545K, | |
| | P2A- | | H1047R | |
| | HER2- | | | |
| | P2A-HER3 | | | |
| pRB-139 | HER2- | Y537C, E380Q, | E542K, | GGGGS |
| | P2A-PI3K- | K303R, Y537S, | H1047, | |
| | P2A- | D538G, Y537N | E545K, | |
| | HER3- | | H1047R | |
| | P2A-ESR1 | | | |
| pRB-140 | HER2- | Y537C, E380Q, | E542K, | GGGGS |
| | P2A-PI3K- | K303R, Y537S, | H1047, | |
| | P2A- | D538G, Y537N | E545K, | |
| | ESR1- | | H1047R | |
| | P2A-HER3 | | | |
| pRB-141 | PI3K-P2A- | Y537C, E380Q, | E542K, | GGGGS |
| | HER2- | K303R, Y537S, | H1047, | |
| | P2A- | D538G, Y537N | E545K, | |
| | HER3- | | H1047R | |
| | P2A-ESR1 | | | |
| pRB-142 | PI3K-P2A- | Y537C, E380Q, | E542K, | GGGGS |
| | HER2- | K303R, Y537S, | H1047, | |
| | P2A- | D538G, Y537N | E545K, | |
| | ESR1- | | H1047R | |
| | P2A-HER3 | | | |
| pRB-143 | HER3- | Y537C, E380Q, | E542K, | GGGGS |
| | P2A- | K303R, Y537S, | H1047, | |
| | ESR1- | D538G, Y537N | E545K, | |
| | P2A- | | H1047R | |
| | HER2- | | | |
| | P2A-PI3K | | | |
| pRB-144 | HER3- | Y537C, E380Q, | E542K, | GGGGS |
| | P2A- | K303R, Y537S, | H1047, | |
| | ESR1- | D538G, Y537N | E545K, | |
| | P2A-PI3K- | | H1047R | |
| | P2A-HER2 | | | |
| pRB-145 | ESR1- | Y537C, E380Q, | E542K, | GGGGS |
| | P2A- | K303R, Y537S, | H1047, | |
| | HER3- | D538G, Y537N | E545K, | |
| | P2A- | | H1047R | |
| | HER2- | | | |
| | P2A-PI3K | | | |
| pRB-146 | ESR1- | Y537C, E380Q, | E542K, | GGGGS |
| | P2A- | K303R, Y537S, | H1047, | |
| | HER3- | D538G, Y537N | E545K, | |
| | P2A-PI3K- | | H1047R | |
| | P2A-HER2 | | | |
| pRB-147 | HER2- | Y537C, E380Q, | E542K, | GGGGS |
| | P2A-PI3K- | K303R, Y537S, | H1047, | |
| | IRES- | D538G, Y537N | E545K, | |
| | HER3- | | H1047R | |
| | P2A-ESR1 | | | |
| pRB-148 | HER2- | Y537C, E380Q, | E542K, | GGGGS |
| | P2A-PI3K- | K303R, Y537S, | H1047, | |
| | IRES- | D538G, Y537N | E545K, | |
| | ESR1- | | H1047R | |
| | P2A-HER3 | | | |
| pRB-149 | PI3K-P2A- | Y537C, E380Q, | E542K, | GGGGS |
| | HER2- | K303R, Y537S, | H1047, | |
| | IRES- | D538G, Y537N | E545K, | |
| | HER3- | | H1047R | |
| | P2A-ESR1 | | | |
| pRB-150 | PI3K-P2A- | Y537C, E380Q, | E542K, | GGGGS |
| | HER2- | K303R, Y537S, | H1047, | |
| | IRES- | D538G, Y537N | E545K, | |
| | ESR1- | | H1047R | |
| | P2A-HER3 | | | |
| pRB-151 | HER3- | Y537C, E380Q, | E542K, | GGGGS |
| | P2A- | K303R, Y537S, | H1047, | |
| | ESR1- | D538G, Y537N | E545K, | |
| | IRES- | | H1047R | |
| | HER2- | | | |
| | P2A-PI3K | | | |
| pRB-152 | HER3- | Y537C, E380Q, | E542K, | GGGGS |
| | P2A- | K303R, Y537S, | H1047, | |
| | ESR1- | D538G, Y537N | E545K, | |
| | IRES- | | H1047R | |
| | PI3K-P2A- | | | |
| | HER2 | | | |
| pRB-153 | ESR1- | Y537C, E380Q, | E542K, | GGGGS |
| | P2A- | K303R, Y537S, | H1047, | |
| | HER3- | D538G, Y537N | E545K, | |
| | IRES- | | H1047R | |
| | HER2- | | | |
| | P2A-PI3K | | | |
| pRB-154 | ESR1- | Y537C, E380Q, | E542K, | GGGGS |
| | P2A- | K303R, Y537S, | H1047, | |
| | HER3- | D538G, Y537N | E545K, | |
| | IRES- | | H1047R | |
| | PI3K-P2A- | | | |
| | HER2 | | | |

In some embodiments, the configuration is selected from the group consisting of pRB-136, pRB-146, pRB-151, and pRB-153.

In some embodiments, nucleic acid sequence has at least 80%, at least 85%, at least 90%, at least 95%, at least 96%, at least 97%, at least 98%, at least 99%, or 100% sequence identity to a nucleic acid sequence selected from the group consisting of SEQ ID NOS: 7-10.

In some embodiments, the coding sequence for a polypeptide construct comprises, in 5′- to 3′-direction: a) a coding sequence for a variant of PI3K comprising one or more molecular alterations selected from E542K, H1047L, E545K, and, wherein said molecular alterations are operably linked to one another by a GGGGS linker; b) a coding sequence for an autoproteolytic peptide P2A; c) a coding sequence for a variant of HER2 comprising its extracellular domain and transmembrane domain; d) a coding sequence for an autoproteolytic peptide P2A; e) a coding sequence for a kinase-inactive variant of HER3; f) a coding sequence for an internal ribosomal entry site (IRES); and g) a coding sequence for a variant of ESR1 comprising one or more molecular alterations selected from H1047R Y537C, E380Q, K303R, Y537S, D538G, and Y537N, wherein said molecular alterations are operably linked to one another by a GGGGS linker.

In some embodiments, the coding sequence of ESR1, PI3K, HER2, and/or HER3 is redesigned and/or optimized for a desired property, such as increased stability, potency, and expression (e.g., translation efficiency), which in turns can maximize the impact of producing, delivering, and administering biotherapeutic. For example, in some embodiments, the coding sequence is optimized for expression at a level higher than the expression level of a reference coding sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure may also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence is optimized for expression in a target host cell through the use of codons optimized for expression. The techniques for the construction of synthetic nucleic acid sequences encoding genes using preferred codons optimal for host cell expression may be determined by computational methods analyzing the commonality of codon usage for encoding native proteins of the host cell genome and their relative abundance by techniques well known in the art. The codon usage database (http://www.kazusa.or.jp/codon) may be used for generation of codon optimized sequences in mammalian cell environments. Furthermore, a variety of software tools are available to convert sequences from one organism to the optimal codon usage for a different host organism such as the JCat Codon Optimization Tool (www.jcat.de), Integrated DNA Technologies (IDT) Codon Optimization Tool (https://www.idtdna.com/CodonOpt) or the Optimizer online codon optimization tool (http://genomes.urv.es/OPTIMIZER). Such synthetic sequences may be constructed by techniques known in the art for the construction of synthetic nucleic acid molecules and may be obtained from a variety of commercial vendors.

In some embodiments, the coding sequence is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Additional information regarding principles, strategies, and methods for use in enhancing RNA stability can be found at, for example, Leppek K. et al., Combinatorial optimization of mRNA structure, stability, and translation for RNA-based therapeutics. bioRxiv. (Preprint). Mar. 30, 2021. doi: 10.1101/2021.03.29.437587.

### Recombinant Cells

The nucleic acid constructs of the present disclosure can be introduced into a host cell to produce a recombinant cell containing the nucleic acid molecule. Accordingly, prokaryotic or eukaryotic cells that contain a nucleic acid construct encoding a modified EEEV genome as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

In some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), a canine kidney cell (MDCK), a buffalo rat liver cell (BRL 3A), a human lung cell (W138), a human liver cell (Hep G2), a mouse mammary tumor (MMT 060562), a TRI cell, a FS4 cell, a Chinese hamster ovary cell (CHO cell), an African green monkey kidney cell (Vero cell), a human A549 cell, a human cervix cell, a human CHMES cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D.E., Available lepidopteran insect cell lines. Methods Mol Biol. 2007;388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimamus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos. 55, Sua1B, 4a-3B, Mos.43, MSQ43, and LSB-AA695BB. In some embodiments, the mosquito cell is a cell of a C6/26 cell line.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

The recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

### Pharmaceutical Compositions

The nucleic acid constructs, recombinant cells, recombinant polypeptides of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of a health condition such as cancer. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. The nucleic acid constructs of the disclosure can be used in a naked form or formulated with a delivery vehicle. Exemplary routes, either using in a free form, e.g., inserted into a nucleic acid, e.g., a vector. For example, as described in greater detail below, a nucleic acid construct as described herein can be used as a vaccine.

In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient.

In some embodiments, the compositions of the disclosure are formulated for the prevention, treatment, or management of a health condition such as cancer. For example, the compositions of the disclosure can be formulated as a prophylactic composition, a therapeutic composition, or a pharmaceutical composition comprising a pharmaceutically acceptable excipient, or a mixture thereof.

For use in a pharmaceutical composition of the disclosure, a nucleic acid, or a recombinant cell as described herein can be formulated into or with delivery vehicles. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, IRL press (1990). Adjuvants other than liposomes and the like are also used and are known in the art. Adjuvants may protect the antigen (e.g., srRNA construct) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

Accordingly, in some embodiments, a composition of the disclosure can include one or more of the following: physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

In some embodiments, the nucleic acid constructs of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

The lipids suitable for the compositions and methods described herein can be cationic lipids, ionizable cationic lipids, anionic lipids, or neutral lipids.

In some embodiments, the LNP of the disclosure can include one or more ionizable lipids. As used herein, the term “ionizable lipid” refers to a lipid that is cationic or becomes ionizable (protonated) as the pH is lowered below the pKa of the ionizable group of the lipid, but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH, such as physiological pH. Permanently cationic lipids such as DOTMA have proven too toxic for clinical use. The ionizable lipid can be present in lipid formulations according to other embodiments, preferably in a ratio of about 30 to about 70 Mol %, in some embodiments, about 30 Mol %, in other embodiments, about 40 Mol %, in other embodiments, about 45 Mol % in other embodiments, about 47.5 Mol % in other embodiments, about 50 Mol %, in still other embodiments, and about 60 Mol % in yet others (“Mol %” means the percentage of the total moles that is of a particular component). The term “about” in this paragraph signifies a plus or minus range of 5 Mol %. DODMA, or 1,2-dioleyloxy-3-dimethylaminopropane, is an ionizable lipid, as is DLin-MC3-DMA or 0-(Z,Z,Z,Z-heptatriaconta-6,9,26,29-tetraen-19-yl)-4-(N,N-dimethylamino) (“MC3”).

Exemplary ionizable lipids suitable for the compositions and methods of the disclosure includes those described in PCT publications WO2020252589A1 and WO2021000041A1, U.S. Pat. Nos. 8,450,298 and 10,844,028, and Love K.T. et al., Proc Natl Acad Sci USA, Feb. 2, 2010 107 (5) 1864-1869, all of which are hereby incorporated by reference in their entirety. Accordingly, in some embodiments, the LNP of the disclosure includes one or more lipid compounds described in Love K.T. et al., 2010 supra, such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200. The structure of C12-200 lipid is known in the art and described in, e.g., U.S. Pat. Nos. 8,450,298 and 10,844,028, which are hereby incorporated by reference in their entirety. In some embodiments the C12-200 is combined with cholesterol, C14-PEG2000, and DOPE. In some embodiments, the C12-200 is combined with DSPC and DMG-PEG2000.

In some embodiments, the LNP of the disclosure includes one or more cationic lipids. Suitable cationic lipids include, but are not limited to, 98N12-5, C12-200, C14-PEG2000, DLin-KC2-DMA (KC2), DLin-MC3-DMA (MC3), XTC, MD1, and 7C1. In some embodiments, the LNP of the disclosure includes one or more neutral lipids. Non-limiting neutral lipids suitable for the compositions and methods of the disclosure include DPSC, DPPC, POPC, DOPE, and S.M. In some embodiments, the LNP of the disclosure includes one or more ionizable lipid compounds described in PCT publications WO2020252589A1 and WO2021000041A1, which are hereby incorporated by reference in their entirety.

A number of other lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of cationic lipids include 98N12-5, C12-200, C14-PEG2000, DLin-KC2-DMA (KC2), DLin-MC3-DMA (MC3), XTC, MD1, 7C1, and a combination of any thereof. Non-limiting examples of neutral lipids include DPSC, DPPC, POPC, DOPE, and S.M. Non-limiting examples of PEG-modified lipids include PEG-DMG, PEG-CerC14, and PEG-CerC20.

In some embodiments, the LNP of the disclosure includes at least one lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). In some embodiments the C12-200 is combined with cholesterol, C14-PEG2000, and DOPE. In some embodiments, the C12-200 is combined with DSPC and DMG-PEG2000.

In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments, the compositions of the disclosure that formulated in a liposome. In some embodiments, the compositions of the disclosure that formulated in a lipid-based nanoparticle (LNP). In some embodiments, the compositions of the disclosure that formulated in a polymer nanoparticle.

As described above, neural lipids, also known as “structural lipids” or “helper lipids” can also be incorporated into lipid formulations and lipid particles in some embodiments. The lipid formulations and lipid particles can include one or more structural lipids at about 10 to 40 Mol % of the composition. Suitable structural lipids support the formation of particles during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides.

Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE).

In another embodiment, the structural lipid can be any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

Stabilizing agents can be included in lipid formulations embodiments to ensure integrity of the mixtures. Stabilizing agents are a class of molecules which disrupt or help form the hydrophobic-hydrophilic interactions among molecules. Suitable Stabilizing agents include, but are not limited to, polysorbate 80 (also known as Tween 80, IUPAC name 2-[2-[3,4-bis(2-hydroxyethoxy)oxolan-2-yl]-2-(2-hydroxyethoxy)ethoxy]ethyl octadec-9-enoate), Myrj52 (Polyoxyethylene (40) stearate), and Brij™ S10 (Polyoxyethylene (10) stearyl ether). Polyethylene glycol conjugated lipids may also be used. The stabilizing agents may be used alone or in combinations with each other.

In some embodiments, the stabilizing agents comprises about 0.1 to 3 Mol % of the overall lipid mixture. In some embodiments, the stabilizing agents comprise about 0.5 to 2.5 Mol % of the overall lipid mixture. In some embodiments, the stabilizing agent is present at greater than 2.5 Mol %. In some embodiments the stabilizing agent is present at 5 Mol %. In some embodiments the stabilizing agent is present at 10 Mol %. In some embodiments, the stabilizing agent is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, and so forth. In other embodiments, the stabilizing agent is 2.6-10 Mol % of the lipid mixture. In other embodiments, the stabilizing agents is present at greater than 10 Mol % of the lipid mixture.

Steroids can also be included in the lipid compositions for certain applications, and lipid particles made therefrom include sterols, such as cholesterol and phytosterol.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having, or who may be at high risk for developing a cancer.

In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant. In some embodiments, the immunogenic compositions are formulated as a biotherapeutic, e.g., vehicle for gene delivery of different molecules with bioactivity. Non-limiting examples of biotherapeutic include cytokines, chemokines, and other soluble immunomodulators, enzymes, peptide and protein agonists, peptide and protein antagonists, hormones, receptors, antibodies and antibody-derivatives, growth factors, transcription factors, and gene silencing/editing molecules. In some embodiments, the pharmaceutical compositions are formulated as an adjuvant.

In some embodiments, the immunogenic compositions are substantially non-immunogenic or minimally immunogenic (e.g. compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intranodal administration, intratumoral administration, intraarticular administration, intravenous administration, subcutaneous administration, intravaginal administration, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS), tris (tromethamine), and HEPES. In these cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sucrose, trehalose, and/or sodium chloride in the composition. In some embodiments, the composition comprises tris and sucrose. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intramuscular administration, intratumoral administration, intranodal administration, intravenous administration, intraperitoneal administration, oral administration, intravaginal, or intra-cranial administration.

### Methods of the Disclosure

Administration of any one of the therapeutic compositions described herein, e.g., nucleic acid constructs, recombinant cells, recombinant polypeptides, and/or pharmaceutical compositions, can be used in the treatment of relevant health conditions, such as cancers. In some embodiments, the nucleic acid constructs, recombinant cells, recombinant polypeptides, and/or pharmaceutical compositions as described herein can be useful for eliciting an immune response in a subject in need thereof. In some embodiments, the nucleic acid constructs, recombinant cells, recombinant polypeptides, and/or pharmaceutical compositions as described herein can be incorporated into therapeutic agents for use in methods of treating a subject who has, who is suspected of having, or who may be at high risk for developing one or more relevant health conditions or diseases. Exemplary health conditions or diseases can include, without limitation, breast cancers. In some embodiments, the subject is a patient under the care of a physician.

Non-limiting examples of breast cancer suitable for the methods of the disclosure include breast ductal cancer, breast lobular cancer, breast undifferentiated cancer, breast lobular sarcoma, breast angiosarcoma, and primary breast lymphoma. Breast cancer may include stage I, stage II, stage IIIA, stage IIIB, stage IIIC and stage IV breast cancer. Breast ductal carcinomas can include invasive carcinoma types, invasive carcinoma in situ with predominant intraglandular components, inflammatory breast cancers, and ductal carcinomas of the breast. Breast ductal carcinomas can include invasive lobular carcinomas with predominant in situ components, invasive lobular carcinomas, and infiltrating lobular carcinomas. Breast cancer may include Paget's disease, extramammary Paget's disease, Paget's disease with intraglandular cancer, and Paget's disease with invasive ductal carcinoma. Breast cancer may include breast neoplasms with histological and hyperstructural heterogeneity (e.g., mixed cell types). Breast cancer can be classified as basal-like, luminal A, luminal B, ERBB2/Her2 + or normal breast-like molecular subtypes.

Accordingly, in one aspect, provided herein are methods for inducing an immune response in a subject in need thereof, the method includes administering to the subject a composition including: a) a nucleic acid construct of the disclosure; b) a recombinant cell of the disclosure; c) a recombinant polypeptide of the disclosure; and/or d) a pharmaceutical composition of the disclosure.

In another aspect, provided herein are methods for preventing and/or treating a health condition in a subject in need thereof, the method includes prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure.

In some embodiments, the health condition is cancer. In some embodiments, the cancer is breast cancer. In some embodiments, the subject has or is suspected of having cancer.

In some embodiments, the disclosed composition is formulated to be compatible with its intended route of administration. For example, the nucleic acid constructs, recombinant cells, recombinant polypeptides, and/or pharmaceutical compositions of the disclosure may be given orally, by inhalation, or through a parenteral route. Examples of parenteral routes of administration include, for example, intramuscular, intratumoral, intraocular, intravenous, intranodal, intradermal, subcutaneous, transdermal (topical), transmucosal, intravaginal, and rectal administration. In some embodiments, the composition is administered intramuscularly. In some embodiments, the composition is administered intratumorally. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates, phosphates, tris, sucrose and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

The therapeutic compositions described herein, e.g., nucleic acid constructs, recombinant cells, recombinant polypeptides, and/or pharmaceutical compositions, can be administered one from one or more times per day to one or more times per week; including once every other day. Treatment of a subject with a therapeutically effective amount of the subject nucleic acid constructs, recombinant cells, recombinant polypeptides, and/or pharmaceutical compositions of the disclosure can include a single treatment or, can include a series of treatments. In some embodiments, the compositions are administered at weekly intervals, e.g., 1 to 2, 2 to 3, or 3 to 4 doses given at 1 to 2, 2 to 3, or 3 to 4 week intervals. This may be followed with an additional administration every 1, 2, 3, or 4 months. In some embodiments, 3 doses can be administered intramuscularly at a 3 to 4 week interval, followed by intramuscular administration every 3 months. Alternatively, the composition can be administered at shorter intervals, e.g., every 8 hours for five days, followed by a rest period of 2 to 14 days, e.g., 9 days, followed by an additional five days of administration every 8 hours. With regard to nucleic acid constructs and recombinant polypeptides, the therapeutically effective amount of a nucleic acid construct or recombinant polypeptide of the disclosure (e.g., an effective dosage) depends on the nucleic acid construct or recombinant polypeptide selected.

As discussed supra, a therapeutically effective amount includes an amount of a therapeutic composition that is sufficient to promote a particular effect when administered to a subject, such as one who has, is suspected of having, or is at risk for a health condition, e.g., a cancer. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease.

A treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the nucleic acid constructs, recombinant cells, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments of the disclosed methods, the subject is a mammal. In some embodiments, the mammal is a human subject.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the nucleic acid constructs, recombinant cells, and/or recombinant polypeptides in the required mount in an appropriate solvent with one or a combination of ingredients enumerated above, as required, followed by filtered sterilization.

When the nucleic acid constructs, recombinant cells, recombinant polypeptides, and/or pharmaceutical compositions are suitably protected, as described above, they may be orally administered, for example, with an inert diluent or an assimilable edible carrier. The nucleic acid constructs, recombinant cells, recombinant polypeptides, and/or pharmaceutical compositions and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein as well as written instructions for making and using the same. In particular, some embodiments of the disclosure provide kits for eliciting an immune response in a subject. Some other embodiments relate to kits for methods of treating cancer in a subject in need thereof. For example, provided herein, in some embodiments, are kits that include one or more of the nucleic acid constructs, recombinant cells, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs, recombinant cells, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs, recombinant cells, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production of the provided nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs, recombinant cells, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs, recombinant cells, and/or recombinant polypeptides of the disclosure in combination with one or more further therapeutic agents formulated together, optionally, in a pharmaceutical composition, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs, recombinant cells, and/or recombinant polypeptides of the disclosure.

In some embodiments, a kit can further include instructions for using the components of the kit to practice the methods disclosed herein For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Mamial (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Construction of EEEV Vectors

This Example describes the experiments performed to construct a base EEEV vector (e.g., without a heterologous gene) that was subsequently used for construction of a EEEV vectors that express a gene or genes of interest (e.g., ESR1 or variants thereof, PI3K or variants thereof, HER2 or a variant thereof, and HER3 or a variant thereof).

The base EEEV vector (i.e. without a heterologous gene of interest) was constructed as follows: The base EEEV vector was synthesized de novo in four ˜4 kb parts (Twist Bioscience) from a reference sequence (Genbank EF151502) with several modifications. Silent mutations G301A, A3550C, G4516A, G5725A, and G7399A were incorporated to eliminate restriction enzyme cut sites. A unique restriction enzyme cut site (SpeI, 5′-A′CTAG,T-3′) was incorporated in place of the coding sequence of the native EEEV structural genes (where the 5′ A matches the location of the structural polyprotein ATG start codon, and the 3′ T matches the location of the structural polyprotein stop codon TAA). A 5′ adaptor sequence (5′-CTGGAGACGTGGAGGAGAACCCTGGACCT-3′; SEQ ID NO: 21) was inserted upstream of the SpeI site, and a 3′ adaptor sequence (5′-GACCGCTACGCCCCAATGACCCGACCAGC-3′; SEQ ID NO: 22) was inserted downstream of the SpeI site for subsequent Gibson Assembly® procedures (Gibson et al., Nat. Methods 6, 343-345, 2009). A bacteriophage T7 RNA polymerase promoter (5′-TAATACGACTCACTATAG-3′; SEQ ID NO: 23) was included upstream of the EEEV genome sequence, and downstream contained a poly (A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 24) followed by a unique restriction enzyme cut site (NotI, 5′-GC′GGCC,GC-3′). The parts were combined in a five-piece Gibson Assembly® reaction: a linearized pYL backbone and the four synthesized fragments to result in the EEEV base vector.

Construction of an EEEV vector containing heterologous genes was carried out as follows: the base EEEV vector was linearized by SpeI digestion. The ESR1, PI3K, HER2, and HER3 variants were codon optimized/refactored for human expression in silico and along with the EMCV IRES, were synthesized de novo (GeneArt, IDT). The synthetic products were amplified using primers which added either 5′ and 3′ adaptor sequences to the ends of the genes, or primers which added P2A sequences and/or sequences of homology to neighboring gene inserts. The digestion product and PCR products were combined by Gibson Assembly® procedure to result in the final vectors.

### Example 2

### In Vitro Evaluation of Modified EEEV Vectors

This Example describes the results of in vitro experiments performed to evaluate expression levels of the synthetic EEEV replicon constructs described in Example 1 above, and to investigate any differential behavior thereof (e.g., replication and protein expression).

In vitro transcription: RNA was prepared by in vitro transcription from a SapI-linearized plasmid template with bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA was then purified using phenol/chloroform extraction, or column purification (Monarch® RNA Cleanup Kit, NEB). RNA concentration was determined by absorbance at 260 nm (Nanodrop, Thermo Fisher Scientific).

Replication: RNA was transformed by electroporation into BHK-21 or Vero cells (e.g., 4D-Nucleofector™, Lonza). At 15-22 hours following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

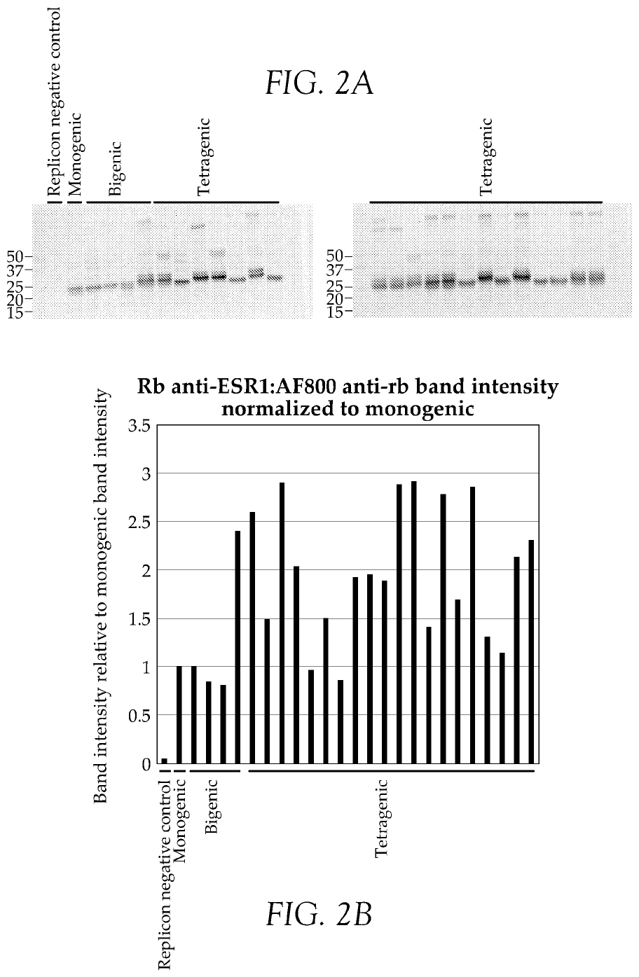

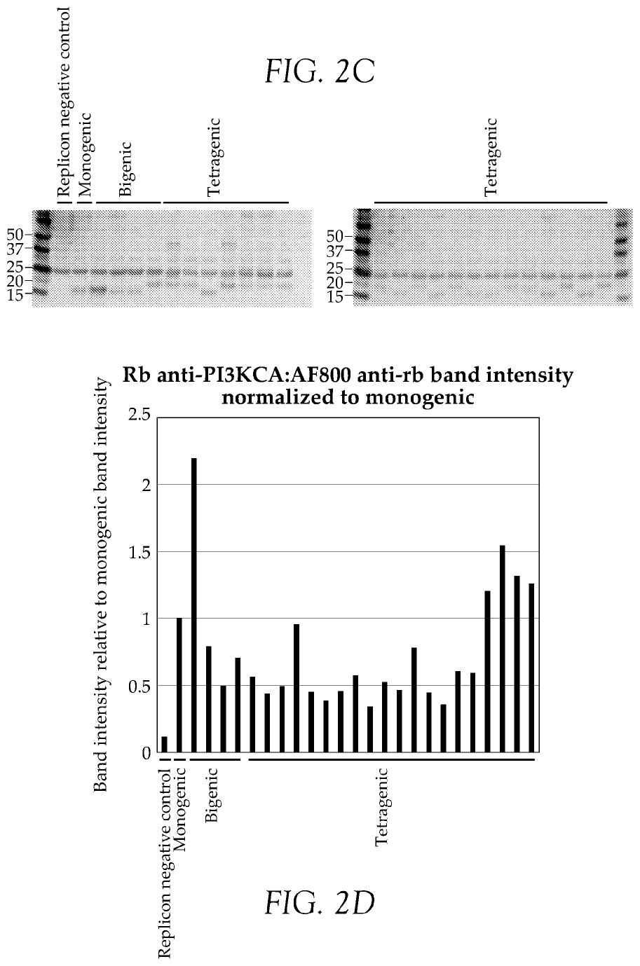

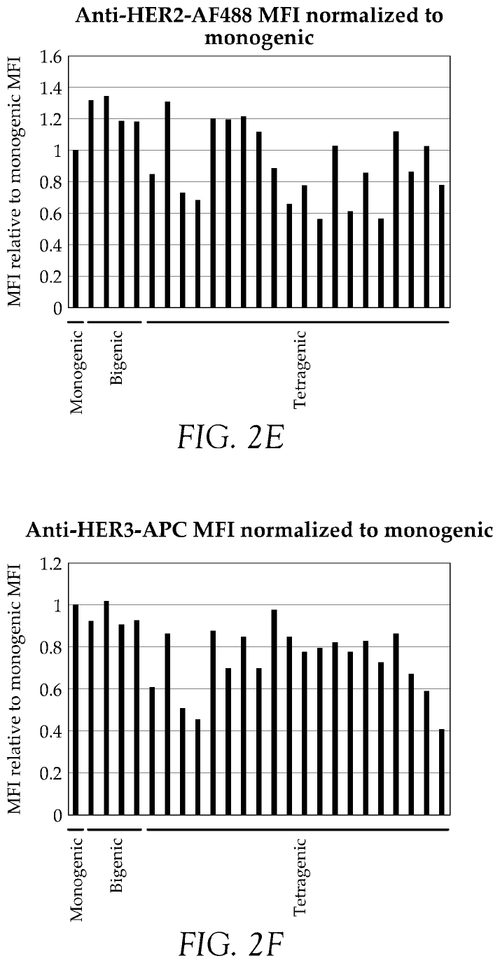

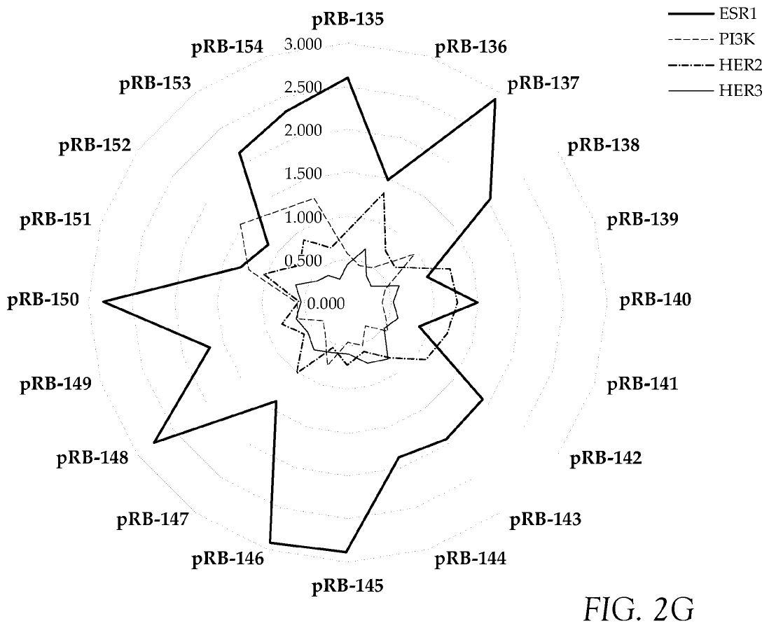

Protein expression: RNA was transformed by electroporation into BHK-21 or Vero cells (e.g., 4D-Nucleofector™, Lonza). ESR1: At 15-22 hours following transformation, the cells were collected and lysed in RIPA buffer. Lysate protein concentration was normalized, then probed in an immunoblot with an anti-ERα rabbit antibody (A300-497A, Bethyl) and imaged using an AF800 conjugated anti-rabbit goat antibody (A32735, Thermo) (FIG. 2A). The fluorescence signal from the cell samples transformed by a synthetic monogenic EEEV replicon expressing ESR1 was used to normalize expression levels to evaluate relative ESR1 expression from the panel of bigenic and tetragenic replicons (FIG. 2B). PI3K: At 15-22 hours following transformation, the cells were collected and lysed in RIPA buffer. Lysate protein concentration was normalized, then probed in an immunoblot with an anti-PI3KCA rabbit antibody (PA587398, Thermo) and imaged using an AF800 conjugated anti-rabbit goat antibody (A32735, Thermo) (FIG. 2C). The fluorescence signal from the cell samples transformed by a synthetic monogenic EEEV replicon expressing PI3K was used to normalize expression levels to evaluate relative PI3K expression from the panel of bigenic and tetragenic replicons (FIG. 2D). HER2: At 15-22 hours following transformation, the cells were fixed and permeabilized (eBioscience™ Foxp3/Transcription Factor Staining Buffer Set, Invitrogen) and stained using an AF488-conjugated anti-HER2 mouse monoclonal antibody (24D2, Biolegend). The mean fluorescence intensity (MFI) of AF488 is used as the readout of HER2 expression. The MFI of cells transformed by a synthetic monogenic EEEV replicon expressing HER2 was used to normalize expression levels to evaluate relative HER2 expression from the panel of bigenic and tetragenic replicons (FIG. 2E). HER3: At 15-22 hours following transformation, the cells were fixed and permeabilized (eBioscience™ Foxp3/Transcription Factor Staining Buffer Set, Invitrogen) and stained using an APC-conjugated anti-HER3 mouse monoclonal antibody (IB4C3, Biolegend). The mean fluorescence intensity (MFI) of APC was used as the readout of HER3 expression. The MFI of cells transformed by a synthetic monogenic EEEV replicon expressing HER3 was used to normalize expression levels to evaluate relative HER3 expression from the panel of bigenic and tetragenic replicons (FIG. 2F). The normalized ESR1, PI3K, HER2, and HER3 expression data from the tetragenic replicons was visualized on a spider graph (FIG. 2G).

### Example 3

### In Vivo Evaluation of Modified EEEV Vectors

This Example describes the results of in vivo experiments performed to evaluate any differential immune responses following vaccination with the synthetic EEEV replicon constructs described herein (e.g., both unformulated and LNP formulated vectors).

In these experiments, synthetic replicon constructs derived from the EEEV strain FL93-939 were designed and subsequently evaluated.

Mice and injections. BALB/c mice were purchased from Charles River Labs, Envigo, or Jackson Laboratories. On day of dosing, between 0.01-10 μg of material was injected intramuscularly either into one or split into both quadricep muscles. Vectors were administered either unformulated in saline, or LNP-formulated. Animals were monitored for body weight and other general observations throughout the course of the study. For immunogenicity studies, animals were dosed on Day 0 only or Day 0 and Day 21.

LNP formulation. Replicon RNA was formulated in lipid nanoparticles using a microfluidics mixer and analyzed for particle size, polydispersity using dynamic light scattering and encapsulation efficiency. Lipids were suspended in ethanol. For L1, RNA was suspended in 10 mM citrate buffer pH 5.0 a concentration of 172 μg/ml, and is mixed at a flow rate of 3:1 (aqueous: organic). For L2, RNA was suspended in 250 mM NaOAc pH 4.0 at a concentration of 82 μg/ml, and is mixed at a flow rate of 3:1 (aqueous: organic).

ELISpot. To measure the magnitude of ESR1-, HER2, and HER3-specific T cell responses, IFNγ ELISpot analysis was performed using Mouse IFNγ ELISpot PLUS Kit (HRP) (MabTech) as per manufacturer's instructions. In brief, splenocytes were isolated and resuspended to a concentration of 5×10⁶ cells/mL in media containing peptides derived from ESR1, HER2, and HER3, PMA/ionomycin as a positive control, or DMSO as a mock stimulation.

### Evaluation of Linkers

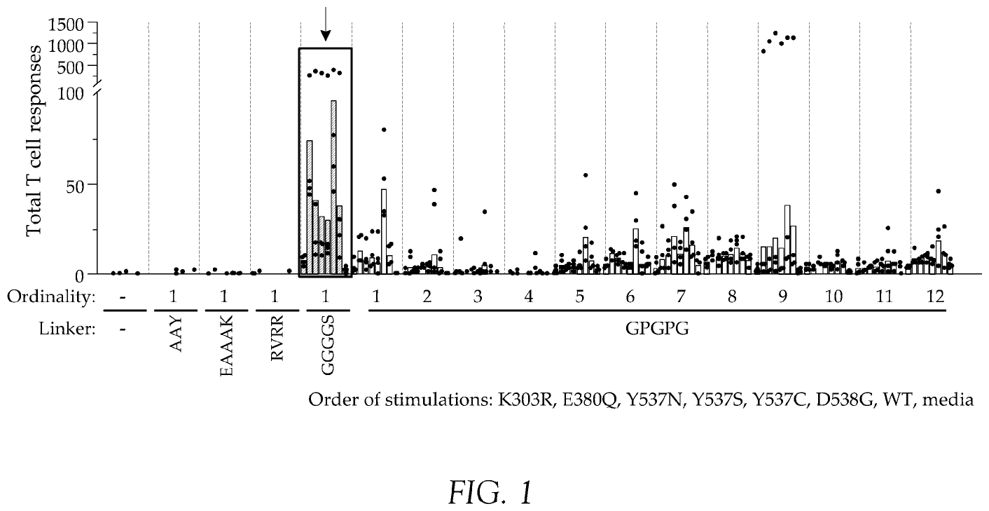

Results of mouse IFNγ detecting ELISpot assay as measured by spot-forming units corresponding to responder splenic T cells 14 days after intramuscular injection of monogenic replicon RNA encoding ESR1 mutations in different ordinalities and inter-connected by different linkers is shown in FIG. 1. AAY, EAAAK, RVRR, GGGGS, and GPGPG linkers that were tested in varying ordinalities in an ESR1 antigen cassette containing the K303R, E380Q, Y537C, Y537S, Y537N, and D538G mutations. Columns for each cassette corresponds to the following stimulation conditions with a single peptide in the order of K303R, E380Q, Y537N, Y537S, Y537C, D538G, wildtype ESR1, and media. The total T cell responses (plotted as counted spot-forming units per million of cells) are shown the y-axis. The GGGGS linker in ordinality 1 produced the most robust T cell responses.

### Evaluation of Number and Ordinality of Genes

Results of mouse IFNγ detecting ELISpot assay as measured by spot-forming units corresponding to responder splenic T cells at Day 35 after two intramuscular injections of replicon RNA encoding ESR1, HER2, and HER3 are shown in FIG. 3. Different constructs, in either monogenic, bigenic or tetragenic form, having different ordinalities and connecting sequences of ESR1, PI3K, HER2, and HER3, were tested in order to determine which configuration of genes in the constructs yielded the most robust T-cell responses upon stimulation. The Y-axis shows the total T cell responses. PI3K responses were not measured in this experiment because it does not form responses in BALB/c mice.

### Evaluation of Ordinality of Genes and Lipid Formulation

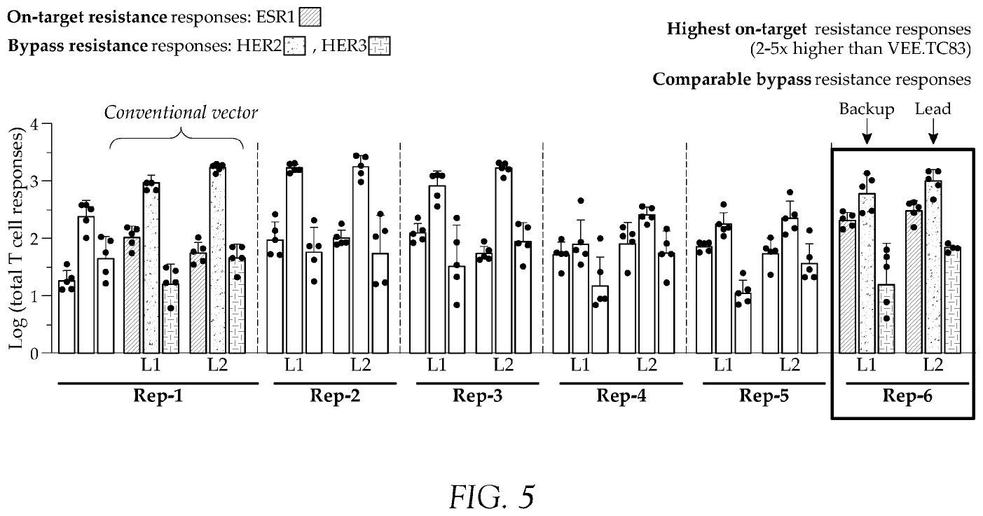

Results of mouse IFNγ detecting ELISpot assay as measured by spot-forming units corresponding to responder splenic T cells at Day 35 after two intramuscular injections of replicon RNA either in saline, or formulated in two different LNP compositions L1 or L2 encoding ESR1, PI3K, HER2, and HER3 are shown in FIG. 5. Different tetragenic constructs, having different replicon vector backbones were tested to determine which RNA replicon vector and formulation yielded the most robust T cell responses upon stimulation.

### Example 4

### Estrogen Receptor Positive Breast Cancer Efficacy

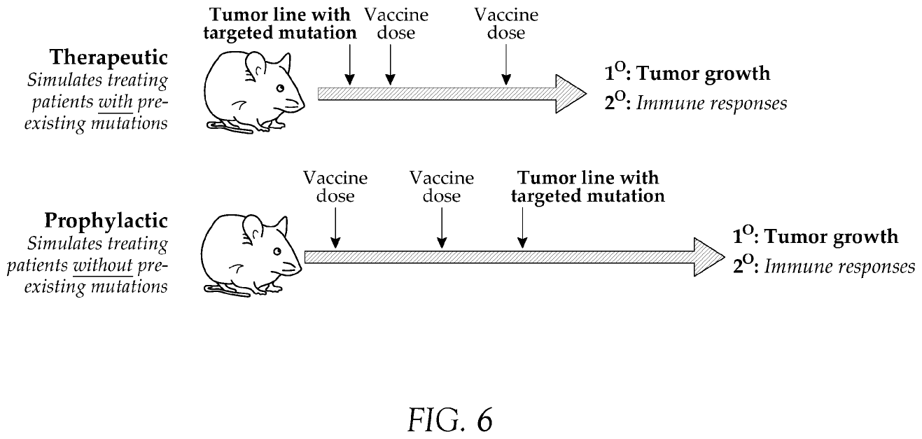

Two efficacy models are shown in FIG. 6 to mimic two clinical scenarios. In the therapeutic model, the tumor cell line expressing a resistance mutation being targeted by the vaccine is implanted first. Vaccination is administered subsequently. This simulates a scenario of treating patients with pre-existing mutations. In the prophylactic model, vaccination is administered prior to implanting the tumor cell line encoding a resistance mutation included in the vaccine. This scenario mimics treating patients prior to the emergence of acquired mutations. Administration of replicon RNA encoding mutation(s) expressed by the tumor cell line should elicit robust T cell responses in mice that will lead to delayed tumor growth. If tumor growth is not delayed, it is likely that the tumor cell line has evolved to lose the targeted mutation, showing that the replicon RNA was able to exert selective pressure by the immune system to lose the activating mutation.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A nucleic acid construct comprising a nucleic acid sequence encoding a modified Eastern Equine Encephalitis virus (EEEV) genome or self-replicating RNA (srRNA), wherein at least a portion of the nucleic acid sequence encoding the viral structural proteins of the modified EEEV genome or srRNA has been replaced by a coding sequence for a polypeptide construct comprising:
a) a coding sequence for estrogen receptor 1 (ESR1) or a variant thereof;
b) a coding sequence for PI3K or a variant thereof;
c) a coding sequence for HER2 or a variant thereof and
d) a coding sequence for HER3 or a variant thereof.

2. The nucleic acid construct of claim 1, wherein the modified EEEV genome or srRNA comprises no nucleic acid sequence encoding viral structural proteins.

3. The nucleic acid construct of claim 1, wherein the nucleic acid sequence encoding the modified EEEV or srRNA is operably linked to a promoter sequence.

4. The nucleic acid construct of claim 1, wherein the coding sequences of (a) through (d) are operably linked to one another within a single open reading frame (ORF).

5. The nucleic acid construct of claim 1, wherein the coding sequences of (a) through (d) are operably linked to one another by one or more connector sequences encoding an autoproteolytic peptide or an internal ribosomal entry site (IRES).

6. The nucleic acid construct of claim 5, wherein the autoproteolytic peptide comprises one or more autoproteolytic cleavage sequences from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

7. The nucleic acid construct of claim 5, wherein the internal ribosomal entry site (IRES) is from a Kaposi's sarcoma-associated herpesvirus (KSHV) IRES, a hepatitis virus IRES, a Pestivirus IRES, a Cripavirus IRES, a Rhopalosiphum padi virus IRES, a fibroblast growth factor IRES, a platelet-derived growth factor IRES, a vascular endothelial growth factor IRES, an insulin-like growth factor IRES, a picornavirus IRES, an encephalomyocarditis virus (EMCV) IRES, a Pim-1 IRES, a p53 IRES, an Apaf-1 IRES, a TDP2 IRES, an L-myc IRES, and a c-myc IRES.

8. The nucleic acid construct of claim 1, wherein at least one of the coding sequences of (a) through (d) comprises one or more molecular alterations.

9. The nucleic acid of claim 8, wherein the one or more molecular alterations are configured into a plurality of alteration cassettes arranged in tandem along the length of the coding sequence.

10. The nucleic acid of claim 9, wherein the plurality of alteration cassettes are operably linked to one another by one or more linkers.

11. The nucleic acid construct of claim 8, wherein the coding for an ESR1 variant in (a) comprises one or more molecular alterations that promote ligand-independent receptor activities.

12. The nucleic acid construct of claim 11, wherein the one or more molecular alterations comprises an activating mutation selected from the group consisting of K303R, E380Q, Y537C, Y537S, Y537N, and D538G.

13. The nucleic acid construct of claim 8, wherein the PI3K variant in (b) comprises one or more molecular alterations that promote ligand-independent receptor activities.

14. The nucleic acid construct of claim 13, wherein the one or more molecular alterations comprises an activating mutation selected from the group consisting of E542K, E545K, H1047L, and H1047R.

15. The nucleic acid construct of claim 1, wherein the HER2 variant in (c) comprises a coding sequence for the extracellular domain and transmembrane domain.

16. The nucleic acid construct of claim 1, wherein the HER3 variant in (d) comprises a coding sequence for a kinase-inactive HER3.

17. The nucleic acid construct of claim 1, wherein the nucleic acid sequence has at least 80%, at least 85%, at least 90%, at least 95%, at least 96%, at least 97%, at least 98%, at least 99%, or 100% sequence identity to a nucleic acid sequence selected from the group consisting of SEQ ID NOS: 7-10.

18. The nucleic acid construct of claim 1, wherein the coding sequence for the polypeptide construct comprises, in 5′- to 3′-direction:
a) a coding sequence for a variant of PI3K comprising one or more activating mutations selected from E542K, H1047L, E545K, and H1047R;
b) a coding sequence for an autoproteolytic peptide P2A;
c) a coding sequence for a variant of HER2 comprising its extracellular domain and transmembrane domain;
d) a coding sequence for an autoproteolytic peptide P2A;
e) a coding sequence for a kinase-inactive variant of HER3;
f) a coding sequence for an internal ribosomal entry site (IRES); and
g) a coding sequence for a variant of ESR1 comprising one or more activating mutations selected from Y537C, E380Q, K303R, Y537S, D538G, and Y537N.

19. A recombinant cell comprising a nucleic acid construct according to claim 1.

20. The recombinant cell of claim 19, wherein the recombinant cell is a mammalian cell or an insect cell.

21. A pharmaceutical composition comprising a pharmaceutically acceptable excipient and:
a nucleic acid construct of claim 1.

22. The pharmaceutical composition of claim 21, wherein the composition is formulated with a delivery vehicle into a delivery system, wherein the delivery system comprises a liposome, a viral replicon particle (VRP), a lipid-based nanoparticle (LNP), a polymer nanoparticle, a physiologic buffer, a microsphere, an immune stimulating complex (ISCOM), a conjugate of bioactive ligand, or a combination of any thereof.

23. The pharmaceutical composition of claim 22, wherein the lipid is present in mass ratio of lipid to RNA from about 100:1 to about 4:1.

24. The pharmaceutical composition of claim 22, wherein the lipid-based nanoparticles have an average diameter of about 25 nm to about 1000 nm.

25. The pharmaceutical composition of claim 21, wherein the composition is formulated as a vaccine.

26. A method for inducing an immune response or treating a health condition in a subject in need thereof, the method comprises administering to the subject a composition comprising a nucleic acid construct of claim 1.

27. The method of claim 26, wherein the method is a method for inducing an immune response.

28. The method of claim 26, wherein the method is a method for treating cancer.

29. The method of claim 28, wherein the cancer is breast cancer.

30. The method of claim 25, wherein the composition is administered to the subject individually as a single therapy (monotherapy) or as a first therapy in combination with at least one additional therapies.


SEQUENCE LISTING (29 sequences)

SEQ ID NO: 1 - protein, 595 aa, synthetic construct [sequence omitted]

SEQ ID NO: 2 - protein, 216 aa, synthetic construct
  REGION 1..216: note = misc_feature - variant of ESR1
MEHLYSMKCKNVVPLCDLLLEMLDAHRLHAPGGGGSPGFVDLTLHDQVHLLQCAWLEILMIGLVWRSGGGGSAANLWPSPLMIKRSKRNSLALSLTADQMVSAGGGGSMEHLYSMKCKNVVPLSDLLLEMLDAHRLHAPGGGGSMEHLYSMKCKNVVPLYGLLLEMLDAHRLHAPGGGGSMEHLYSMKCKNVVPLNDLLLEMLDAHRLHAPGGGGS

SEQ ID NO: 3 - protein, 1068 aa, synthetic construct
  REGION 1..1068: note = misc_feature - full-length amino acid sequence of PI3K
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 4 - protein, 147 aa, synthetic construct
  REGION 1..147: note = misc_feature - variant of PI3K
MDKEQLKAISTRDPLSKITEQEKDFLWSHRHYGGGGSEQEALEYFMKQMNDALHGGWTTKMDWIFHTIKGGGGSQLKAISTRDPLSEITKQEKDFLWSHRHYCVTGGGGSEQEALEYFMKQMNDARHGGWTTKMDWIFHTIKGGGGS

SEQ ID NO: 5 - protein, 668 aa, synthetic construct
  REGION 1..668: note = misc_feature - truncated HER2 variant
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSPLTSIISAVVGILLVVVLGVVFGILIKRRQQKIRK

SEQ ID NO: 6 - protein, 1342 aa, synthetic construct
  REGION 1..1342: note = misc_feature - kinase-inactive HER3 variant
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 7 - RNA, 15857 nt, synthetic construct
  misc_feature 1..15857: note = pRB-136
  source 1..15857: mol_type = other RNA
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaaaagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagaacttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagaactggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatggacaaagagcagctgaaggcaatcagcacccgggatcctctgagcaagatcaccgagcaagagaaggacttcctgtggtcccacagacattatggcggcggaggctctgaacaagaggccctggaatactttatgaagcagatgaacgacgccctgcacggcggctggacaacaaagatggattggatcttccacaccatcaaaggtggcggaggctcccagctgaaagctatctctaccagagatcccctgtccgagatcacgaagcaagaaaaagatttcctttggagccaccggcactactgcgttacaggtggtggcggaagcgagcaagaagctctcgaatatttcatgaagcaaatgaatgatgccaggcatggcggatggaccaccaaaatggactggatttttcatacgatcaaaggcggtggcggcagcggaagcggcgccacaaatttcagcctgctgaaacaggccggcgacgtggaagagaatcctggacctatggaactggccgctctgtgcagatggggactgcttcttgcacttcttccacctggcgccgctagcacacaagtgtgcacaggcaccgacatgaagctgagactgcctgcctctcctgagacacacctggacatgctgagacacctgtaccagggttgtcaggtggtgcagggcaacctggaactgacctacctgcctacaaacgccagcctgagctttctgcaggacatccaagaggtgcagggatacgtgctgatcgcccacaatcaagtgcgacaggtgcccctgcagagactgagaatcgttagaggcacccagctgttcgaggacaattatgccctggccgtgctggacaacggcgaccctcttaacaataccacacctgtgacaggcgcctctccaggcggactgagagaactgcaactgagaagcctgaccgagatcctgaaaggcggagtgctgatccagagaaaccctcagctgtgctaccaggacaccatcctgtggaaggacatcttccacaagaacaaccagctggccctgacactgatcgacaccaacagaagcagagcctgccatccttgcagccccatgtgcaagggatctagatgttggggcgagagcagcgaggattgccagagcctgacaagaacagtgtgtgccggcggatgtgccagatgtaaaggccctctgcctaccgattgctgccatgagcaatgtgccgctggctgtacaggccctaagcactctgattgtctggcctgcctgcacttcaaccactctggaatctgcgagctgcactgccctgctctggtcacctacaacaccgacaccttcgagagcatgcccaatcctgagggcagatacacctttggcgccagctgtgtgaccgcctgtccttacaattacctgagcaccgatgtgggcagctgcaccctcgtgtgtcctctgcataatcaagaagtgaccgccgaggacggcacccagagatgcgagaagtgtagcaagccttgcgccagagtgtgttacggcctcggcatggaacatctgagagaagtgcgggccgtgaccagcgccaatatccaagagtttgccggctgcaagaagatctttggcagcctggccttcctgcctgagagctttgatggcgatcctgccagcaatactgcccctctgcagcctgaacagctccaggtgttcgagacactggaagagatcaccggctacctgtacatcagcgcctggcctgatagcctgcctgatctgagcgtgttccagaacctgcaagtgatccggggcagaatcctgcacaacggcgcctattctctgacactgcaaggcctgggaatcagctggctgggcctgagatctctgagagagcttggatctggcctggctctgatccaccataacacccacctgtgcttcgtgcacaccgtgccttgggaccagctgtttagaaatccccatcaggccctgctgcacaccgccaatagacctgaggatgagtgtgttggcgaaggcctggcttgtcaccaactgtgtgcaagaggacactgttggggccctggacctacacagtgcgtgaactgctctcagttcctgagaggccaagagtgcgtggaagagtgtagagtgcttcaaggactgccccgcgagtacgtgaacgccagacattgtctgccttgtcaccctgagtgccagcctcagaatggcagcgtgacatgttttggccctgaggccgaccagtgtgtggcctgtgctcactacaaggaccctccattctgcgtggccagatgtcctagcggcgtgaagccagatctgtcctacatgcccatctggaagttccccgatgaggaaggcgcttgccagccttgtcctatcaactgcacacacagccctctgaccagcatcatctctgccgttgtgggaatcctgctggtggtggtgctgggcgttgtgttcggaatcctgatcaagcggcggcagcagaaaatccggaagggaagcggcgccacaaatttcagcctgctgaaacaggccggcgacgtggaagagaatcctggacctatgagagccaacgacgccctgcaggtcctgggacttctgttttctctggctagaggcagcgaagtgggcaactctcaggctgtgtgtcctggcacactgaatggcctgtctgtgacaggcgacgccgagaaccagtaccagacactgtacaagctgtacgagagatgcgaggtggtcatgggcaacctggaaatcgtgctgaccggccacaatgccgatctgagcttcctgcagtggatccgggaagtgacaggatacgtgctggtggccatgaacgagttcagcaccctgcctctgcctaacctgagagtcgttagaggcacccaggtgtacgacggcaagttcgccatcttcgtgatgctgaactacaacaccaacagctctcacgccctgcggcagctgagactgacacagctgacagagattctgtctggcggcgtgtacatcgagaagaacgacaagctgtgccacatggacaccatcgactggcgggacatcgtgcgggatagagatgccgagatcgtggtcaaggacaacggcagaagctgccctccttgccacgaagtgtgcaagggaagatgttggggccctggcagcgaggattgtcagaccctgaccaagacaatctgcgcccctcagtgcaatggccactgcttcggccctaatcctaaccagtgctgccacgatgaatgcgctggcggatgtagcggccctcaggatacagattgcttcgcctgcagacacttcaacgatagcggcgcctgtgtgcctagatgtcctcagcctctggtgtacaacaagctgacctttcagctggaacccaatcctcacaccaagtaccagtacggcggagtgtgtgtggccagctgtcctcacaatttcgtggtggatcagaccagctgtgtgcgggcctgtcctcctgacaagatggaagtggacaagaacggcctgaagatgtgcgagccttgtggcggactgtgccctaaagcttgtgaaggcacaggcagcggcagcagatttcagaccgtggacagcagcaacatcgacggcttcgtgaactgcaccaagatcctgggcaatctggacttcctgatcaccggcctgaatggcgacccttggcacaagattccagctctggaccccgagaagctgaacgtgttcagaaccgtgcgggaaatcaccggctacctgaacatccagtcctggcctccacacatgcacaacttcagcgtgttctccaacctgaccaccatcggcggcagatccctgtacaatagaggcttcagcctgctgatcatgaagaacctgaatgtgaccagcctgggcttcagaagcctgaaagagatcagcgccggcagaatctacatcagcgccaacagacagctgtgctaccaccactctctgaattggaccaaggtgctgagaggccccaccgaggaaagactggacatcaagcacaacagacccagacgggattgcgtggccgagggaaaagtctgcgatcctctgtgttctagcggcggctgttggggtccaggaccaggacaatgcctgagctgcagaaattacagccgcggaggcgtgtgcgtgacccactgcaattttctgaacggcgagcccagagaattcgcccacgaggccgagtgttttagctgtcaccctgagtgccagcctatggaaggcaccgccacatgtaatggcagcggctctgatacttgtgcccagtgcgcccactttagagatggccctcactgtgtgtctagctgcccacatggcgtgctgggagccaagggacctatctacaagtaccccgacgtgcagaacgagtgcagaccatgccacgagaattgcacacagggatgcaagggccccgagctgcaagattgtctgggacagacactggtgctgatcggcaagacacacctgacaatggccctgacagtgatcgccggactggtggtcatctttatgatgctcggcggcaccttcctgtactggcggggaagaagaatccagaacaagcgggccatgcggagatacctggaaagaggcgagagcatcgagcccctggatccttctgagaaggccaacaaagtgctggcccggatcttcaaagagacagagctgcggaagctgaaggtgctcggaagcggagtgtttggcacagtgcacaaaggcgtgtggatccctgagggcgagtccatcaagatccccgtgtgcatcaaagtgatcgaggacaagagcggcaggcagagcttccaggccgtgacagatcatatgctggccatcggatctctggatcacgcccatatcgtcagactgctgggcctgtgtccaggatctagcctgcagctcgtgacacagtatctgcctctgggatctctgctggaccacgttcgacaacatagaggcgctctgggaccccagctgctgctgaattggggagtgcagatcgccaagggcatgtactacctggaagaacacggcatggtgcacagaaacctggccgccagaaatgtgctgctcaagtctcctagtcaggtgcaggtcgccgatttcggagtggctgatctcctgcctcctgatgacaaacagctgctgtactccgaggccaagacacccatcaagtggatggccctggaatctatccacttcggcaagtacacccaccagagcgacgtgtggtcttacggcgtgacagtgtgggagctgatgacatttggagccgagccttatgccggcctgagactggctgaagtgcccgatctgctggaaaaaggggaaagactcgcccagcctcagatctgcaccatcgatgtgtacatggtcatggtcaagtgctggatgatcgacgagaacatcaggcccacctttaaagagctggccaacgagtttacccggatggccagggatcctcctagatacctcgtgatcaagagagagagcggcccaggcattgcacctggacctgaacctcacggactgaccaacaagaaactggaagaggtggaactggaacccgagctggacctggatctcgatctggaagccgaggaagataacctggccaccacaacactgggctctgcactgtctctgcctgtgggcaccctgaatagacctagaggaagccagagcctgctgtcccctagcagcggctacatgcccatgaatcagggaaatctgggcgagagctgtcaagagtctgccgtgtctggcagcagcgaaagatgccctagacctgtgtctctgcaccccatgcctagaggatgtctggcctctgaatctagcgagggccacgtgaccggaagcgaagccgaactgcaagagaaagtctccatgtgccggtccagaagccgcagcagatctcctagaccaagaggcgatagcgcctaccactctcagagacactcactgctgacccctgtgacacctctgtctccacctggcctcgaagaggaagatgtgaacggctacgtgatgcccgacactcacctgaagggcacacctagctctagagagggcacactgtctagcgtgggactgtcctctgtgctgggaaccgaagaagaggacgaggacgaagagtacgagtacatgaaccggcggagaaggcactccccgcctcatcctccaagaccaagctctctcgaagaactgggctacgagtatatggacgtgggcagcgatctgtctgcctctctggggtctacacagagctgtccactgcaccctgtgcctatcatgcctacagccggcaccacacctgatgaggactatgagtatatgaatcggcagcgcgacggcggaggacctggcggagattatgctgctatgggagcctgtccagccagcgagcagggctatgaggaaatgagagcctttcaaggcccaggccaccaggctcctcatgtgcattacgccagactgaaaaccctgcggtccctggaagccaccgacagcgccttcgataaccctgactactggcacagcagactgttccccaaggccaacgctcagagaacctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggaacacctgtacagcatgaagtgcaagaacgtggtgcccctgtgcgacctgctgctggaaatgctggatgcccatagactgcatgctccaggtggcggcggatctcctggatttgtggatctgacactgcacgaccaagtgcatctgctgcagtgcgcctggctggaaatcctgatgatcggcctcgtttggagatctggcggaggcggatctgctgccaatctgtggccttctccactgatgatcaagcggagcaagcggaactctctggccctgtctctgacagccgatcagatggtttctgcaggcggcggaggcagcatggaacatctctactctatgaagtgtaaaaacgtcgtccctctcagcgacttgctccttgagatgctcgacgctcacagacttcatgcacctggcggtggcggctctatggaacacctttattcaatgaagtgcaaaaatgttgtgccgctctacggcctcctcctcgaaatgttggacgcacataggcttcacgctcccggtggcggtggttctatggaacatctctatagtatgaagtgcaagaatgtcgtcccgctgaacgatctgcttttggagatgttggatgctcacaggttgcatgcccctggcggcggtggatcttgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttc

SEQ ID NO: 8 - RNA, 15333 nt, synthetic construct
  misc_feature 1..15333: note = pRB-146
  source 1..15333: mol_type = other RNA
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaaaagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagaacttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagaactggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatggaacacctgtacagcatgaagtgcaagaacgtggtgcccctgtgcgacctgctgctggaaatgctggatgcccatagactgcatgctccaggtggcggcggatctcctggatttgtggatctgacactgcacgaccaagtgcatctgctgcagtgcgcctggctggaaatcctgatgatcggcctcgtttggagatctggcggaggcggatctgctgccaatctgtggccttctccactgatgatcaagcggagcaagcggaactctctggccctgtctctgacagccgatcagatggtttctgcaggcggcggaggcagcatggaacatctctactctatgaagtgtaaaaacgtcgtccctctcagcgacttgctccttgagatgctcgacgctcacagacttcatgcacctggcggtggcggctctatggaacacctttattcaatgaagtgcaaaaatgttgtgccgctctacggcctcctcctcgaaatgttggacgcacataggcttcacgctcccggtggcggtggttctatggaacatctctatagtatgaagtgcaagaatgtcgtcccgctgaacgatctgcttttggagatgttggatgctcacaggttgcatgcccctggcggcggtggatctggaagcggcgccacaaatttcagcctgctgaaacaggccggcgacgtggaagagaatcctggacctatgagagccaacgacgccctgcaggtcctgggacttctgttttctctggctagaggcagcgaagtgggcaactctcaggctgtgtgtcctggcacactgaatggcctgtctgtgacaggcgacgccgagaaccagtaccagacactgtacaagctgtacgagagatgcgaggtggtcatgggcaacctggaaatcgtgctgaccggccacaatgccgatctgagcttcctgcagtggatccgggaagtgacaggatacgtgctggtggccatgaacgagttcagcaccctgcctctgcctaacctgagagtcgttagaggcacccaggtgtacgacggcaagttcgccatcttcgtgatgctgaactacaacaccaacagctctcacgccctgcggcagctgagactgacacagctgacagagattctgtctggcggcgtgtacatcgagaagaacgacaagctgtgccacatggacaccatcgactggcgggacatcgtgcgggatagagatgccgagatcgtggtcaaggacaacggcagaagctgccctccttgccacgaagtgtgcaagggaagatgttggggccctggcagcgaggattgtcagaccctgaccaagacaatctgcgcccctcagtgcaatggccactgcttcggccctaatcctaaccagtgctgccacgatgaatgcgctggcggatgtagcggccctcaggatacagattgcttcgcctgcagacacttcaacgatagcggcgcctgtgtgcctagatgtcctcagcctctggtgtacaacaagctgacctttcagctggaacccaatcctcacaccaagtaccagtacggcggagtgtgtgtggccagctgtcctcacaatttcgtggtggatcagaccagctgtgtgcgggcctgtcctcctgacaagatggaagtggacaagaacggcctgaagatgtgcgagccttgtggcggactgtgccctaaagcttgtgaaggcacaggcagcggcagcagatttcagaccgtggacagcagcaacatcgacggcttcgtgaactgcaccaagatcctgggcaatctggacttcctgatcaccggcctgaatggcgacccttggcacaagattccagctctggaccccgagaagctgaacgtgttcagaaccgtgcgggaaatcaccggctacctgaacatccagtcctggcctccacacatgcacaacttcagcgtgttctccaacctgaccaccatcggcggcagatccctgtacaatagaggcttcagcctgctgatcatgaagaacctgaatgtgaccagcctgggcttcagaagcctgaaagagatcagcgccggcagaatctacatcagcgccaacagacagctgtgctaccaccactctctgaattggaccaaggtgctgagaggccccaccgaggaaagactggacatcaagcacaacagacccagacgggattgcgtggccgagggaaaagtctgcgatcctctgtgttctagcggcggctgttggggtccaggaccaggacaatgcctgagctgcagaaattacagccgcggaggcgtgtgcgtgacccactgcaattttctgaacggcgagcccagagaattcgcccacgaggccgagtgttttagctgtcaccctgagtgccagcctatggaaggcaccgccacatgtaatggcagcggctctgatacttgtgcccagtgcgcccactttagagatggccctcactgtgtgtctagctgcccacatggcgtgctgggagccaagggacctatctacaagtaccccgacgtgcagaacgagtgcagaccatgccacgagaattgcacacagggatgcaagggccccgagctgcaagattgtctgggacagacactggtgctgatcggcaagacacacctgacaatggccctgacagtgatcgccggactggtggtcatctttatgatgctcggcggcaccttcctgtactggcggggaagaagaatccagaacaagcgggccatgcggagatacctggaaagaggcgagagcatcgagcccctggatccttctgagaaggccaacaaagtgctggcccggatcttcaaagagacagagctgcggaagctgaaggtgctcggaagcggagtgtttggcacagtgcacaaaggcgtgtggatccctgagggcgagtccatcaagatccccgtgtgcatcaaagtgatcgaggacaagagcggcaggcagagcttccaggccgtgacagatcatatgctggccatcggatctctggatcacgcccatatcgtcagactgctgggcctgtgtccaggatctagcctgcagctcgtgacacagtatctgcctctgggatctctgctggaccacgttcgacaacatagaggcgctctgggaccccagctgctgctgaattggggagtgcagatcgccaagggcatgtactacctggaagaacacggcatggtgcacagaaacctggccgccagaaatgtgctgctcaagtctcctagtcaggtgcaggtcgccgatttcggagtggctgatctcctgcctcctgatgacaaacagctgctgtactccgaggccaagacacccatcaagtggatggccctggaatctatccacttcggcaagtacacccaccagagcgacgtgtggtcttacggcgtgacagtgtgggagctgatgacatttggagccgagccttatgccggcctgagactggctgaagtgcccgatctgctggaaaaaggggaaagactcgcccagcctcagatctgcaccatcgatgtgtacatggtcatggtcaagtgctggatgatcgacgagaacatcaggcccacctttaaagagctggccaacgagtttacccggatggccagggatcctcctagatacctcgtgatcaagagagagagcggcccaggcattgcacctggacctgaacctcacggactgaccaacaagaaactggaagaggtggaactggaacccgagctggacctggatctcgatctggaagccgaggaagataacctggccaccacaacactgggctctgcactgtctctgcctgtgggcaccctgaatagacctagaggaagccagagcctgctgtcccctagcagcggctacatgcccatgaatcagggaaatctgggcgagagctgtcaagagtctgccgtgtctggcagcagcgaaagatgccctagacctgtgtctctgcaccccatgcctagaggatgtctggcctctgaatctagcgagggccacgtgaccggaagcgaagccgaactgcaagagaaagtctccatgtgccggtccagaagccgcagcagatctcctagaccaagaggcgatagcgcctaccactctcagagacactcactgctgacccctgtgacacctctgtctccacctggcctcgaagaggaagatgtgaacggctacgtgatgcccgacactcacctgaagggcacacctagctctagagagggcacactgtctagcgtgggactgtcctctgtgctgggaaccgaagaagaggacgaggacgaagagtacgagtacatgaaccggcggagaaggcactccccgcctcatcctccaagaccaagctctctcgaagaactgggctacgagtatatggacgtgggcagcgatctgtctgcctctctggggtctacacagagctgtccactgcaccctgtgcctatcatgcctacagccggcaccacacctgatgaggactatgagtatatgaatcggcagcgcgacggcggaggacctggcggagattatgctgctatgggagcctgtccagccagcgagcagggctatgaggaaatgagagcctttcaaggcccaggccaccaggctcctcatgtgcattacgccagactgaaaaccctgcggtccctggaagccaccgacagcgccttcgataaccctgactactggcacagcagactgttccccaaggccaacgctcagagaaccggaagcggcgccacaaatttcagcctgctgaaacaggccggcgacgtggaagagaatcctggacctatggacaaagagcagctgaaggcaatcagcacccgggatcctctgagcaagatcaccgagcaagagaaggacttcctgtggtcccacagacattatggcggcggaggctctgaacaagaggccctggaatactttatgaagcagatgaacgacgccctgcacggcggctggacaacaaagatggattggatcttccacaccatcaaaggtggcggaggctcccagctgaaagctatctctaccagagatcccctgtccgagatcacgaagcaagaaaaagatttcctttggagccaccggcactactgcgttacaggtggtggcggaagcgagcaagaagctctcgaatatttcatgaagcaaatgaatgatgccaggcatggcggatggaccaccaaaatggactggatttttcatacgatcaaaggcggtggcggcagcggaagcggcgccacaaatttcagcctgctgaaacaggccggcgacgtggaagagaatcctggacctatggaactggccgctctgtgcagatggggactgcttcttgcacttcttccacctggcgccgctagcacacaagtgtgcacaggcaccgacatgaagctgagactgcctgcctctcctgagacacacctggacatgctgagacacctgtaccagggttgtcaggtggtgcagggcaacctggaactgacctacctgcctacaaacgccagcctgagctttctgcaggacatccaagaggtgcagggatacgtgctgatcgcccacaatcaagtgcgacaggtgcccctgcagagactgagaatcgttagaggcacccagctgttcgaggacaattatgccctggccgtgctggacaacggcgaccctcttaacaataccacacctgtgacaggcgcctctccaggcggactgagagaactgcaactgagaagcctgaccgagatcctgaaaggcggagtgctgatccagagaaaccctcagctgtgctaccaggacaccatcctgtggaaggacatcttccacaagaacaaccagctggccctgacactgatcgacaccaacagaagcagagcctgccatccttgcagccccatgtgcaagggatctagatgttggggcgagagcagcgaggattgccagagcctgacaagaacagtgtgtgccggcggatgtgccagatgtaaaggccctctgcctaccgattgctgccatgagcaatgtgccgctggctgtacaggccctaagcactctgattgtctggcctgcctgcacttcaaccactctggaatctgcgagctgcactgccctgctctggtcacctacaacaccgacaccttcgagagcatgcccaatcctgagggcagatacacctttggcgccagctgtgtgaccgcctgtccttacaattacctgagcaccgatgtgggcagctgcaccctcgtgtgtcctctgcataatcaagaagtgaccgccgaggacggcacccagagatgcgagaagtgtagcaagccttgcgccagagtgtgttacggcctcggcatggaacatctgagagaagtgcgggccgtgaccagcgccaatatccaagagtttgccggctgcaagaagatctttggcagcctggccttcctgcctgagagctttgatggcgatcctgccagcaatactgcccctctgcagcctgaacagctccaggtgttcgagacactggaagagatcaccggctacctgtacatcagcgcctggcctgatagcctgcctgatctgagcgtgttccagaacctgcaagtgatccggggcagaatcctgcacaacggcgcctattctctgacactgcaaggcctgggaatcagctggctgggcctgagatctctgagagagcttggatctggcctggctctgatccaccataacacccacctgtgcttcgtgcacaccgtgccttgggaccagctgtttagaaatccccatcaggccctgctgcacaccgccaatagacctgaggatgagtgtgttggcgaaggcctggcttgtcaccaactgtgtgcaagaggacactgttggggccctggacctacacagtgcgtgaactgctctcagttcctgagaggccaagagtgcgtggaagagtgtagagtgcttcaaggactgccccgcgagtacgtgaacgccagacattgtctgccttgtcaccctgagtgccagcctcagaatggcagcgtgacatgttttggccctgaggccgaccagtgtgtggcctgtgctcactacaaggaccctccattctgcgtggccagatgtcctagcggcgtgaagccagatctgtcctacatgcccatctggaagttccccgatgaggaaggcgcttgccagccttgtcctatcaactgcacacacagccctctgaccagcatcatctctgccgttgtgggaatcctgctggtggtggtgctgggcgttgtgttcggaatcctgatcaagcggcggcagcagaaaatccggaagtgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttc

SEQ ID NO: 9 - RNA, 15857 nt, synthetic construct
  misc_feature 1..15857: note = pRB-151
  source 1..15857: mol_type = other RNA
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaaaagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagaacttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagaactggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatgagagccaacgacgccctgcaggtcctgggacttctgttttctctggctagaggcagcgaagtgggcaactctcaggctgtgtgtcctggcacactgaatggcctgtctgtgacaggcgacgccgagaaccagtaccagacactgtacaagctgtacgagagatgcgaggtggtcatgggcaacctggaaatcgtgctgaccggccacaatgccgatctgagcttcctgcagtggatccgggaagtgacaggatacgtgctggtggccatgaacgagttcagcaccctgcctctgcctaacctgagagtcgttagaggcacccaggtgtacgacggcaagttcgccatcttcgtgatgctgaactacaacaccaacagctctcacgccctgcggcagctgagactgacacagctgacagagattctgtctggcggcgtgtacatcgagaagaacgacaagctgtgccacatggacaccatcgactggcgggacatcgtgcgggatagagatgccgagatcgtggtcaaggacaacggcagaagctgccctccttgccacgaagtgtgcaagggaagatgttggggccctggcagcgaggattgtcagaccctgaccaagacaatctgcgcccctcagtgcaatggccactgcttcggccctaatcctaaccagtgctgccacgatgaatgcgctggcggatgtagcggccctcaggatacagattgcttcgcctgcagacacttcaacgatagcggcgcctgtgtgcctagatgtcctcagcctctggtgtacaacaagctgacctttcagctggaacccaatcctcacaccaagtaccagtacggcggagtgtgtgtggccagctgtcctcacaatttcgtggtggatcagaccagctgtgtgcgggcctgtcctcctgacaagatggaagtggacaagaacggcctgaagatgtgcgagccttgtggcggactgtgccctaaagcttgtgaaggcacaggcagcggcagcagatttcagaccgtggacagcagcaacatcgacggcttcgtgaactgcaccaagatcctgggcaatctggacttcctgatcaccggcctgaatggcgacccttggcacaagattccagctctggaccccgagaagctgaacgtgttcagaaccgtgcgggaaatcaccggctacctgaacatccagtcctggcctccacacatgcacaacttcagcgtgttctccaacctgaccaccatcggcggcagatccctgtacaatagaggcttcagcctgctgatcatgaagaacctgaatgtgaccagcctgggcttcagaagcctgaaagagatcagcgccggcagaatctacatcagcgccaacagacagctgtgctaccaccactctctgaattggaccaaggtgctgagaggccccaccgaggaaagactggacatcaagcacaacagacccagacgggattgcgtggccgagggaaaagtctgcgatcctctgtgttctagcggcggctgttggggtccaggaccaggacaatgcctgagctgcagaaattacagccgcggaggcgtgtgcgtgacccactgcaattttctgaacggcgagcccagagaattcgcccacgaggccgagtgttttagctgtcaccctgagtgccagcctatggaaggcaccgccacatgtaatggcagcggctctgatacttgtgcccagtgcgcccactttagagatggccctcactgtgtgtctagctgcccacatggcgtgctgggagccaagggacctatctacaagtaccccgacgtgcagaacgagtgcagaccatgccacgagaattgcacacagggatgcaagggccccgagctgcaagattgtctgggacagacactggtgctgatcggcaagacacacctgacaatggccctgacagtgatcgccggactggtggtcatctttatgatgctcggcggcaccttcctgtactggcggggaagaagaatccagaacaagcgggccatgcggagatacctggaaagaggcgagagcatcgagcccctggatccttctgagaaggccaacaaagtgctggcccggatcttcaaagagacagagctgcggaagctgaaggtgctcggaagcggagtgtttggcacagtgcacaaaggcgtgtggatccctgagggcgagtccatcaagatccccgtgtgcatcaaagtgatcgaggacaagagcggcaggcagagcttccaggccgtgacagatcatatgctggccatcggatctctggatcacgcccatatcgtcagactgctgggcctgtgtccaggatctagcctgcagctcgtgacacagtatctgcctctgggatctctgctggaccacgttcgacaacatagaggcgctctgggaccccagctgctgctgaattggggagtgcagatcgccaagggcatgtactacctggaagaacacggcatggtgcacagaaacctggccgccagaaatgtgctgctcaagtctcctagtcaggtgcaggtcgccgatttcggagtggctgatctcctgcctcctgatgacaaacagctgctgtactccgaggccaagacacccatcaagtggatggccctggaatctatccacttcggcaagtacacccaccagagcgacgtgtggtcttacggcgtgacagtgtgggagctgatgacatttggagccgagccttatgccggcctgagactggctgaagtgcccgatctgctggaaaaaggggaaagactcgcccagcctcagatctgcaccatcgatgtgtacatggtcatggtcaagtgctggatgatcgacgagaacatcaggcccacctttaaagagctggccaacgagtttacccggatggccagggatcctcctagatacctcgtgatcaagagagagagcggcccaggcattgcacctggacctgaacctcacggactgaccaacaagaaactggaagaggtggaactggaacccgagctggacctggatctcgatctggaagccgaggaagataacctggccaccacaacactgggctctgcactgtctctgcctgtgggcaccctgaatagacctagaggaagccagagcctgctgtcccctagcagcggctacatgcccatgaatcagggaaatctgggcgagagctgtcaagagtctgccgtgtctggcagcagcgaaagatgccctagacctgtgtctctgcaccccatgcctagaggatgtctggcctctgaatctagcgagggccacgtgaccggaagcgaagccgaactgcaagagaaagtctccatgtgccggtccagaagccgcagcagatctcctagaccaagaggcgatagcgcctaccactctcagagacactcactgctgacccctgtgacacctctgtctccacctggcctcgaagaggaagatgtgaacggctacgtgatgcccgacactcacctgaagggcacacctagctctagagagggcacactgtctagcgtgggactgtcctctgtgctgggaaccgaagaagaggacgaggacgaagagtacgagtacatgaaccggcggagaaggcactccccgcctcatcctccaagaccaagctctctcgaagaactgggctacgagtatatggacgtgggcagcgatctgtctgcctctctggggtctacacagagctgtccactgcaccctgtgcctatcatgcctacagccggcaccacacctgatgaggactatgagtatatgaatcggcagcgcgacggcggaggacctggcggagattatgctgctatgggagcctgtccagccagcgagcagggctatgaggaaatgagagcctttcaaggcccaggccaccaggctcctcatgtgcattacgccagactgaaaaccctgcggtccctggaagccaccgacagcgccttcgataaccctgactactggcacagcagactgttccccaaggccaacgctcagagaaccggaagcggcgccacaaatttcagcctgctgaaacaggccggcgacgtggaagagaatcctggacctatggaacacctgtacagcatgaagtgcaagaacgtggtgcccctgtgcgacctgctgctggaaatgctggatgcccatagactgcatgctccaggtggcggcggatctcctggatttgtggatctgacactgcacgaccaagtgcatctgctgcagtgcgcctggctggaaatcctgatgatcggcctcgtttggagatctggcggaggcggatctgctgccaatctgtggccttctccactgatgatcaagcggagcaagcggaactctctggccctgtctctgacagccgatcagatggtttctgcaggcggcggaggcagcatggaacatctctactctatgaagtgtaaaaacgtcgtccctctcagcgacttgctccttgagatgctcgacgctcacagacttcatgcacctggcggtggcggctctatggaacacctttattcaatgaagtgcaaaaatgttgtgccgctctacggcctcctcctcgaaatgttggacgcacataggcttcacgctcccggtggcggtggttctatggaacatctctatagtatgaagtgcaagaatgtcgtcccgctgaacgatctgcttttggagatgttggatgctcacaggttgcatgcccctggcggcggtggatcttgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggaactggccgctctgtgcagatggggactgcttcttgcacttcttccacctggcgccgctagcacacaagtgtgcacaggcaccgacatgaagctgagactgcctgcctctcctgagacacacctggacatgctgagacacctgtaccagggttgtcaggtggtgcagggcaacctggaactgacctacctgcctacaaacgccagcctgagctttctgcaggacatccaagaggtgcagggatacgtgctgatcgcccacaatcaagtgcgacaggtgcccctgcagagactgagaatcgttagaggcacccagctgttcgaggacaattatgccctggccgtgctggacaacggcgaccctcttaacaataccacacctgtgacaggcgcctctccaggcggactgagagaactgcaactgagaagcctgaccgagatcctgaaaggcggagtgctgatccagagaaaccctcagctgtgctaccaggacaccatcctgtggaaggacatcttccacaagaacaaccagctggccctgacactgatcgacaccaacagaagcagagcctgccatccttgcagccccatgtgcaagggatctagatgttggggcgagagcagcgaggattgccagagcctgacaagaacagtgtgtgccggcggatgtgccagatgtaaaggccctctgcctaccgattgctgccatgagcaatgtgccgctggctgtacaggccctaagcactctgattgtctggcctgcctgcacttcaaccactctggaatctgcgagctgcactgccctgctctggtcacctacaacaccgacaccttcgagagcatgcccaatcctgagggcagatacacctttggcgccagctgtgtgaccgcctgtccttacaattacctgagcaccgatgtgggcagctgcaccctcgtgtgtcctctgcataatcaagaagtgaccgccgaggacggcacccagagatgcgagaagtgtagcaagccttgcgccagagtgtgttacggcctcggcatggaacatctgagagaagtgcgggccgtgaccagcgccaatatccaagagtttgccggctgcaagaagatctttggcagcctggccttcctgcctgagagctttgatggcgatcctgccagcaatactgcccctctgcagcctgaacagctccaggtgttcgagacactggaagagatcaccggctacctgtacatcagcgcctggcctgatagcctgcctgatctgagcgtgttccagaacctgcaagtgatccggggcagaatcctgcacaacggcgcctattctctgacactgcaaggcctgggaatcagctggctgggcctgagatctctgagagagcttggatctggcctggctctgatccaccataacacccacctgtgcttcgtgcacaccgtgccttgggaccagctgtttagaaatccccatcaggccctgctgcacaccgccaatagacctgaggatgagtgtgttggcgaaggcctggcttgtcaccaactgtgtgcaagaggacactgttggggccctggacctacacagtgcgtgaactgctctcagttcctgagaggccaagagtgcgtggaagagtgtagagtgcttcaaggactgccccgcgagtacgtgaacgccagacattgtctgccttgtcaccctgagtgccagcctcagaatggcagcgtgacatgttttggccctgaggccgaccagtgtgtggcctgtgctcactacaaggaccctccattctgcgtggccagatgtcctagcggcgtgaagccagatctgtcctacatgcccatctggaagttccccgatgaggaaggcgcttgccagccttgtcctatcaactgcacacacagccctctgaccagcatcatctctgccgttgtgggaatcctgctggtggtggtgctgggcgttgtgttcggaatcctgatcaagcggcggcagcagaaaatccggaagggaagcggcgccacaaatttcagcctgctgaaacaggccggcgacgtggaagagaatcctggacctatggacaaagagcagctgaaggcaatcagcacccgggatcctctgagcaagatcaccgagcaagagaaggacttcctgtggtcccacagacattatggcggcggaggctctgaacaagaggccctggaatactttatgaagcagatgaacgacgccctgcacggcggctggacaacaaagatggattggatcttccacaccatcaaaggtggcggaggctcccagctgaaagctatctctaccagagatcccctgtccgagatcacgaagcaagaaaaagatttcctttggagccaccggcactactgcgttacaggtggtggcggaagcgagcaagaagctctcgaatatttcatgaagcaaatgaatgatgccaggcatggcggatggaccaccaaaatggactggatttttcatacgatcaaaggcggtggcggcagctgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttc

SEQ ID NO: 10 - RNA, 15857 nt, synthetic construct
  misc_feature 1..15857: note = pRB-153
  source 1..15857: mol_type = other RNA
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaaaagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagaacttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagaactggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatggaacacctgtacagcatgaagtgcaagaacgtggtgcccctgtgcgacctgctgctggaaatgctggatgcccatagactgcatgctccaggtggcggcggatctcctggatttgtggatctgacactgcacgaccaagtgcatctgctgcagtgcgcctggctggaaatcctgatgatcggcctcgtttggagatctggcggaggcggatctgctgccaatctgtggccttctccactgatgatcaagcggagcaagcggaactctctggccctgtctctgacagccgatcagatggtttctgcaggcggcggaggcagcatggaacatctctactctatgaagtgtaaaaacgtcgtccctctcagcgacttgctccttgagatgctcgacgctcacagacttcatgcacctggcggtggcggctctatggaacacctttattcaatgaagtgcaaaaatgttgtgccgctctacggcctcctcctcgaaatgttggacgcacataggcttcacgctcccggtggcggtggttctatggaacatctctatagtatgaagtgcaagaatgtcgtcccgctgaacgatctgcttttggagatgttggatgctcacaggttgcatgcccctggcggcggtggatctggaagcggcgccacaaatttcagcctgctgaaacaggccggcgacgtggaagagaatcctggacctatgagagccaacgacgccctgcaggtcctgggacttctgttttctctggctagaggcagcgaagtgggcaactctcaggctgtgtgtcctggcacactgaatggcctgtctgtgacaggcgacgccgagaaccagtaccagacactgtacaagctgtacgagagatgcgaggtggtcatgggcaacctggaaatcgtgctgaccggccacaatgccgatctgagcttcctgcagtggatccgggaagtgacaggatacgtgctggtggccatgaacgagttcagcaccctgcctctgcctaacctgagagtcgttagaggcacccaggtgtacgacggcaagttcgccatcttcgtgatgctgaactacaacaccaacagctctcacgccctgcggcagctgagactgacacagctgacagagattctgtctggcggcgtgtacatcgagaagaacgacaagctgtgccacatggacaccatcgactggcgggacatcgtgcgggatagagatgccgagatcgtggtcaaggacaacggcagaagctgccctccttgccacgaagtgtgcaagggaagatgttggggccctggcagcgaggattgtcagaccctgaccaagacaatctgcgcccctcagtgcaatggccactgcttcggccctaatcctaaccagtgctgccacgatgaatgcgctggcggatgtagcggccctcaggatacagattgcttcgcctgcagacacttcaacgatagcggcgcctgtgtgcctagatgtcctcagcctctggtgtacaacaagctgacctttcagctggaacccaatcctcacaccaagtaccagtacggcggagtgtgtgtggccagctgtcctcacaatttcgtggtggatcagaccagctgtgtgcgggcctgtcctcctgacaagatggaagtggacaagaacggcctgaagatgtgcgagccttgtggcggactgtgccctaaagcttgtgaaggcacaggcagcggcagcagatttcagaccgtggacagcagcaacatcgacggcttcgtgaactgcaccaagatcctgggcaatctggacttcctgatcaccggcctgaatggcgacccttggcacaagattccagctctggaccccgagaagctgaacgtgttcagaaccgtgcgggaaatcaccggctacctgaacatccagtcctggcctccacacatgcacaacttcagcgtgttctccaacctgaccaccatcggcggcagatccctgtacaatagaggcttcagcctgctgatcatgaagaacctgaatgtgaccagcctgggcttcagaagcctgaaagagatcagcgccggcagaatctacatcagcgccaacagacagctgtgctaccaccactctctgaattggaccaaggtgctgagaggccccaccgaggaaagactggacatcaagcacaacagacccagacgggattgcgtggccgagggaaaagtctgcgatcctctgtgttctagcggcggctgttggggtccaggaccaggacaatgcctgagctgcagaaattacagccgcggaggcgtgtgcgtgacccactgcaattttctgaacggcgagcccagagaattcgcccacgaggccgagtgttttagctgtcaccctgagtgccagcctatggaaggcaccgccacatgtaatggcagcggctctgatacttgtgcccagtgcgcccactttagagatggccctcactgtgtgtctagctgcccacatggcgtgctgggagccaagggacctatctacaagtaccccgacgtgcagaacgagtgcagaccatgccacgagaattgcacacagggatgcaagggccccgagctgcaagattgtctgggacagacactggtgctgatcggcaagacacacctgacaatggccctgacagtgatcgccggactggtggtcatctttatgatgctcggcggcaccttcctgtactggcggggaagaagaatccagaacaagcgggccatgcggagatacctggaaagaggcgagagcatcgagcccctggatccttctgagaaggccaacaaagtgctggcccggatcttcaaagagacagagctgcggaagctgaaggtgctcggaagcggagtgtttggcacagtgcacaaaggcgtgtggatccctgagggcgagtccatcaagatccccgtgtgcatcaaagtgatcgaggacaagagcggcaggcagagcttccaggccgtgacagatcatatgctggccatcggatctctggatcacgcccatatcgtcagactgctgggcctgtgtccaggatctagcctgcagctcgtgacacagtatctgcctctgggatctctgctggaccacgttcgacaacatagaggcgctctgggaccccagctgctgctgaattggggagtgcagatcgccaagggcatgtactacctggaagaacacggcatggtgcacagaaacctggccgccagaaatgtgctgctcaagtctcctagtcaggtgcaggtcgccgatttcggagtggctgatctcctgcctcctgatgacaaacagctgctgtactccgaggccaagacacccatcaagtggatggccctggaatctatccacttcggcaagtacacccaccagagcgacgtgtggtcttacggcgtgacagtgtgggagctgatgacatttggagccgagccttatgccggcctgagactggctgaagtgcccgatctgctggaaaaaggggaaagactcgcccagcctcagatctgcaccatcgatgtgtacatggtcatggtcaagtgctggatgatcgacgagaacatcaggcccacctttaaagagctggccaacgagtttacccggatggccagggatcctcctagatacctcgtgatcaagagagagagcggcccaggcattgcacctggacctgaacctcacggactgaccaacaagaaactggaagaggtggaactggaacccgagctggacctggatctcgatctggaagccgaggaagataacctggccaccacaacactgggctctgcactgtctctgcctgtgggcaccctgaatagacctagaggaagccagagcctgctgtcccctagcagcggctacatgcccatgaatcagggaaatctgggcgagagctgtcaagagtctgccgtgtctggcagcagcgaaagatgccctagacctgtgtctctgcaccccatgcctagaggatgtctggcctctgaatctagcgagggccacgtgaccggaagcgaagccgaactgcaagagaaagtctccatgtgccggtccagaagccgcagcagatctcctagaccaagaggcgatagcgcctaccactctcagagacactcactgctgacccctgtgacacctctgtctccacctggcctcgaagaggaagatgtgaacggctacgtgatgcccgacactcacctgaagggcacacctagctctagagagggcacactgtctagcgtgggactgtcctctgtgctgggaaccgaagaagaggacgaggacgaagagtacgagtacatgaaccggcggagaaggcactccccgcctcatcctccaagaccaagctctctcgaagaactgggctacgagtatatggacgtgggcagcgatctgtctgcctctctggggtctacacagagctgtccactgcaccctgtgcctatcatgcctacagccggcaccacacctgatgaggactatgagtatatgaatcggcagcgcgacggcggaggacctggcggagattatgctgctatgggagcctgtccagccagcgagcagggctatgaggaaatgagagcctttcaaggcccaggccaccaggctcctcatgtgcattacgccagactgaaaaccctgcggtccctggaagccaccgacagcgccttcgataaccctgactactggcacagcagactgttccccaaggccaacgctcagagaacctgagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggaactggccgctctgtgcagatggggactgcttcttgcacttcttccacctggcgccgctagcacacaagtgtgcacaggcaccgacatgaagctgagactgcctgcctctcctgagacacacctggacatgctgagacacctgtaccagggttgtcaggtggtgcagggcaacctggaactgacctacctgcctacaaacgccagcctgagctttctgcaggacatccaagaggtgcagggatacgtgctgatcgcccacaatcaagtgcgacaggtgcccctgcagagactgagaatcgttagaggcacccagctgttcgaggacaattatgccctggccgtgctggacaacggcgaccctcttaacaataccacacctgtgacaggcgcctctccaggcggactgagagaactgcaactgagaagcctgaccgagatcctgaaaggcggagtgctgatccagagaaaccctcagctgtgctaccaggacaccatcctgtggaaggacatcttccacaagaacaaccagctggccctgacactgatcgacaccaacagaagcagagcctgccatccttgcagccccatgtgcaagggatctagatgttggggcgagagcagcgaggattgccagagcctgacaagaacagtgtgtgccggcggatgtgccagatgtaaaggccctctgcctaccgattgctgccatgagcaatgtgccgctggctgtacaggccctaagcactctgattgtctggcctgcctgcacttcaaccactctggaatctgcgagctgcactgccctgctctggtcacctacaacaccgacaccttcgagagcatgcccaatcctgagggcagatacacctttggcgccagctgtgtgaccgcctgtccttacaattacctgagcaccgatgtgggcagctgcaccctcgtgtgtcctctgcataatcaagaagtgaccgccgaggacggcacccagagatgcgagaagtgtagcaagccttgcgccagagtgtgttacggcctcggcatggaacatctgagagaagtgcgggccgtgaccagcgccaatatccaagagtttgccggctgcaagaagatctttggcagcctggccttcctgcctgagagctttgatggcgatcctgccagcaatactgcccctctgcagcctgaacagctccaggtgttcgagacactggaagagatcaccggctacctgtacatcagcgcctggcctgatagcctgcctgatctgagcgtgttccagaacctgcaagtgatccggggcagaatcctgcacaacggcgcctattctctgacactgcaaggcctgggaatcagctggctgggcctgagatctctgagagagcttggatctggcctggctctgatccaccataacacccacctgtgcttcgtgcacaccgtgccttgggaccagctgtttagaaatccccatcaggccctgctgcacaccgccaatagacctgaggatgagtgtgttggcgaaggcctggcttgtcaccaactgtgtgcaagaggacactgttggggccctggacctacacagtgcgtgaactgctctcagttcctgagaggccaagagtgcgtggaagagtgtagagtgcttcaaggactgccccgcgagtacgtgaacgccagacattgtctgccttgtcaccctgagtgccagcctcagaatggcagcgtgacatgttttggccctgaggccgaccagtgtgtggcctgtgctcactacaaggaccctccattctgcgtggccagatgtcctagcggcgtgaagccagatctgtcctacatgcccatctggaagttccccgatgaggaaggcgcttgccagccttgtcctatcaactgcacacacagccctctgaccagcatcatctctgccgttgtgggaatcctgctggtggtggtgctgggcgttgtgttcggaatcctgatcaagcggcggcagcagaaaatccggaagggaagcggcgccacaaatttcagcctgctgaaacaggccggcgacgtggaagagaatcctggacctatggacaaagagcagctgaaggcaatcagcacccgggatcctctgagcaagatcaccgagcaagagaaggacttcctgtggtcccacagacattatggcggcggaggctctgaacaagaggccctggaatactttatgaagcagatgaacgacgccctgcacggcggctggacaacaaagatggattggatcttccacaccatcaaaggtggcggaggctcccagctgaaagctatctctaccagagatcccctgtccgagatcacgaagcaagaaaaagatttcctttggagccaccggcactactgcgttacaggtggtggcggaagcgagcaagaagctctcgaatatttcatgaagcaaatgaatgatgccaggcatggcggatggaccaccaaaatggactggatttttcatacgatcaaaggcggtggcggcagctgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttc

SEQ ID NO: 11 - protein, 31 aa, synthetic construct
  REGION 1..31: note = misc_feature - ESR1 portion
MEHLYSMKCKNVVPLCDLLLEMLDAHRLHAP

SEQ ID NO: 12 - protein, 31 aa, synthetic construct
  REGION 1..31: note = misc_feature - ESR1 portion
PGFVDLTLHDQVHLLQCAWLEILMIGLVWRS

SEQ ID NO: 13 - protein, 31 aa, synthetic construct
  REGION 1..31: note = misc_feature - ESR1 portion
AANLWPSPLMIKRSKRNSLALSLTADQMVSA

SEQ ID NO: 14 - protein, 31 aa, synthetic construct
  REGION 1..31: note = misc_feature - ESR1 portion
MEHLYSMKCKNVVPLSDLLLEMLDAHRLHAP

SEQ ID NO: 15 - protein, 31 aa, synthetic construct
  REGION 1..31: note = misc_feature - ESR1 portion
MEHLYSMKCKNVVPLYGLLLEMLDAHRLHAP

SEQ ID NO: 16 - protein, 31 aa, synthetic construct
  REGION 1..31: note = misc_feature - ESR1 portion
MEHLYSMKCKNVVPLNDLLLEMLDAHRLHAP

SEQ ID NO: 17 - protein, 32 aa, synthetic construct
  REGION 1..32: note = misc_feature - PI3K portion
MDKEQLKAISTRDPLSKITEQEKDFLWSHRHY

SEQ ID NO: 18 - protein, 32 aa, synthetic construct
  REGION 1..32: note = misc_feature - PI3K portion
EQEALEYFMKQMNDALHGGWTTKMDWIFHTIK

SEQ ID NO: 19 - protein, 31 aa, synthetic construct
  REGION 1..31: note = misc_feature - PI3K portion
QLKAISTRDPLSEITKQEKDFLWSHRHYCVT

SEQ ID NO: 20 - protein, 32 aa, synthetic construct
  REGION 1..32: note = misc_feature - PI3K portion
EQEALEYFMKQMNDARHGGWTTKMDWIFHTIK

SEQ ID NO: 21 - DNA, 29 bp, synthetic construct
  misc_feature 1..29: note = 5-prime adaptor sequence
  source 1..29: mol_type = other DNA
ctggagacgtggaggagaaccctggacct

SEQ ID NO: 22 - DNA, 29 bp, synthetic construct
  misc_feature 1..29: note = 3-prime adaptor sequence
  source 1..29: mol_type = other DNA
gaccgctacgccccaatgacccgaccagc

SEQ ID NO: 23 - DNA, 18 bp, synthetic construct
  misc_feature 1..18: note = bacteriophage T7 RNA polymerase promoter
  source 1..18: mol_type = other DNA
taatacgactcactatag

SEQ ID NO: 24 - DNA, 29 bp, synthetic construct
  misc_feature 1..29: note = T7 terminator sequence
  source 1..29: mol_type = other DNA
aacccctctctaaacggaggggttttttt

SEQ ID NO: 25 - skipped

SEQ ID NO: 26 - protein, 5 aa, synthetic construct
  REGION 1..5: note = misc_feature - Peptide linker
EAAAK

SEQ ID NO: 27 - protein, 4 aa, synthetic construct
  REGION 1..4: note = misc_feature - Peptide linker
RVRR

SEQ ID NO: 28 - protein, 5 aa, synthetic construct
  REGION 1..5: note = misc_feature - Peptide linker
GGGGS

SEQ ID NO: 29 - protein, 5 aa, synthetic construct
  REGION 1..5: note = misc_feature - Peptide linker
GPGPG